(19) United States
(12) Patent Application Publication
(10) Pub. No.: US 2021/0137960 A1
(43) Pub. Date: May 13, 2021

(54) COMPOSITIONS AND METHODS FOR INHIBITION OF NUCLEAR-PENETRATING ANTIBODIES

(71) Applicant: Yale University, New Haven, CT (US)

(72) Inventors: James E. Hansen, Guilford, CT (US); Zahra Rattray, New Haven, CT (US)

(21) Appl. No.: 16/967,109

(22) PCT Filed: Feb. 1, 2019
(86) PCT No.: PCT/US2019/016299

§ 371 Date: Aug. 3, 2020

Related U.S. Application Data
(60) Provisional application No. 62/625,213, filed on Feb. 1, 2018.

(51) Int. Cl. A61K 31/7048; A61P 37/06; G01N 33/564; A61K 31/366; A61K 31/517; A61K 31/575; C07K 16/44; C12N 15/113

(52) U.S. Cl. CPC A61K 31/7048; A61P 37/06; G01N 33/564; A61K 31/366; A61K 31/517; C07K 2317/24; C07K 16/44; C12N 15/113; C12N 2310/14; C12N 2310/141; A61K 31/575

(57) ABSTRACT

Compositions and methods of treating autoimmune diseases by administering a subject in need thereof an effective amount of an inhibitor of the importin pathway are also provided. Typically, the autoimmune disease has one or more symptoms or pathologies dependent on or otherwise caused by nuclear penetrating antibodies, for example, nuclear penetrating autoantibodies. In specific embodiments, the autoimmune disease is scleroderma or a form of lupus, for example systemic lupus erythematosus. In preferred embodiments, the inhibitor of the importin pathway is a macrocyclic lactone such as an avermectin or a milbemycin. Compositions, formulations, and dosage forms including an effective amount of the macrocyclic lactones to reduce nuclear localization of a nuclear penetrating antibody are also provided. The compositions can be employed in the disclosed methods. Exemplary dosages ranges and dosage regimens are also provided.

### CROSS-REFERENCE TO RELATED APPLICATION

This application claims benefit of U.S. Provisional Application No. 62/625,213, filed Feb. 1, 2018, which, is hereby incorporated herein by reference in its entirety.

### REFERENCE TO THE SEQUENCE LISTING

The Sequence Listing submitted as a text file named “YU_7364_PCT_ST25.txt,” having a size of 109,684 bytes is hereby incorporated by reference pursuant to 37 C.F.R. § 1.52(e)(5).

### FIELD OF THE INVENTION

This invention is generally related to compositions and methods of use thereof for reducing nuclear translocation of nuclear penetrating antibodies and the treatment of diseases and disorders associated thereof.

### BACKGROUND OF THE INVENTION

Aberrant production of autoantibodies reactive against self-antigens results in inflammation and tissue damage that is characteristic of autoimmune diseases such as systemic lupus erythematosus (SLE), scleroderma, Sjogren's syndrome, Hashimoto's thyroiditis, multiple sclerosis, and many others. While most antibodies are targeted to extracellular antigens such as cell surface receptors or circulating factors, a select subset of autoantibodies has the unusual ability to penetrate live cells where they can target intracellular antigens. For example, some antinuclear antibodies (ANAs) penetrate live cells and localize to nuclei, and cause functional perturbations in autoimmune disease (Yanase and Madaio, in Autoimmune Reactions, S. Paul, Ed. (Humana Press, Totowa, N.J., 1999), pp. 293-304; Rhodes and Isenberg, Trends Immunol 38, 916-926 (2017); Ying-Chyi et al., Eur J Immunol 38, 3178-3190 (2008)). Anti-dsDNA antibodies are highly specific for systemic lupus erythematosus (SLE), and elevated titers are detected in ˜70% of SLE patients, compared to 0.5% in healthy individuals or those presenting with other autoimmune disorders (e.g. rheumatoid arthritis) (Rahman and Isenberg, N Engl J Med 358, 929-939 (2008); Isenberg et al., Arthritis Rheum 28, 999-1007 (1985)). The specific contributions of anti-dsDNA antibodies to lupus pathophysiology are unknown, but these antibodies are included in the ACR (American College of Rheumatology) and SLICC (Systemic Lupus International Collaborating Clinics) SLE classification criteria, and are associated with dermatological and renal manifestations of lupus (Yu et al., J Autoimmun 48-49, 10-3 (2014)).

In particular, multiple cell and nuclear-penetrating lupus anti-DNA autoantibodies have been identified that are believed to contribute to the pathophysiology of SLE (and likely other autoimmune diseases) and in some cases to have the potential to be used as drug delivery vehicles or as single agents designed to perturb intracellular processes such as DNA repair (Noble et al., Nat Rev Rheumatol 12, 429-43 (2016)). For example, the murine anti-DNA autoantibody 3E10, isolated from the MRL/lpr lupus mouse model, has been shown to penetrate live cell nuclei, to localize to tumors due to its affinity for DNA released by dying cancer cells, and to inhibit DNA repair and thereby selectively kill cancer cells with defects in homology-directed repair (HDR) of DNA double-strand breaks (Hansen et al., Sci Transl Med 4(157):157ra142. doi: 10.1126/scitranslmed.3004385 (2012); Noble et al., Cancer Res 75, 2285-91 (2015); Weisbart et al., Sci Rep 5:12022. doi: 10.1038/srep12022. (2015)).

The mechanisms of cellular internalization by autoantibodies are diverse. Some are taken into cells through electrostatic interactions or FcR-mediated endocytosis, while others utilize mechanisms based on association with cell surface myosin or calreticulin, followed by endocytosis (Ying-Chyi et al., Eur J Immunol 38, 3178-3190 (2008), Yanase et al., J Clin Invest 100, 25-31 (1997)). 3E10 penetrates cells in an Fc-independent mechanism (as evidenced by the ability of 3E10 fragments lacking an Fc to penetrate cells) but requires presence of the nucleoside transporter ENT2 (Weisbart et al., Sci Rep 5:12022. doi: 10.1038/srep12022. (2015), Zack et al., J Immunol 157, 2082-2088 (1996), Hansen et al., J Biol Chem 282, 20790-20793 (2007)). In each of the above scenarios, although the method for crossing the cell membrane has been identified, the mechanism by which autoantibodies including 3E10 localize to cell nuclei remains elusive.

Thus, it is an object of the invention to identify the mechanism of nuclear localization of 3E10 and other nuclear penetrating antibodies, and to provide compositions and methods of use thereof, including diagnostic and therapeutic strategies, stemming therefrom.

### SUMMARY OF THE INVENTION

A putative bipartite classical nuclear localizing sequence (NLS) has been identified in the 3E10 variable region of the light chain (VL). This sequence is relatively conserved across a panel of known nuclear-localizing anti-DNA autoantibodies, humanized forms thereof and fragments thereof. These findings implicated the importin nuclear transport pathway in the nuclear localization of nuclear penetrating antibodies. Studies confirmed that inhibition of the importin pathway indeed does inhibit nuclear localization of nuclear penetrating antibodies.

Nuclear penetrating antibodies have been connected to various autoimmune disorders such as systemic lupus erythematosus and scleroderma. Thus, methods of treating an autoimmune disorder including administering to a subject in need thereof an effective amount of an inhibitor of the importin pathway are provided. The inhibitor is typically administrated in an amount that reduces nuclear localization of nuclear penetrating antibodies in the subject. For example, the inhibitor of the importin pathway can inhibit importin-α and/or importin-β, or a structure or function of the nuclear pore. In some embodiments, the inhibitor of the importin pathway inhibits one or more of importin-4, importin-5, importin-7, importin-8, importin-9, importin-11, importin-13, importin-α1, importin-α2, importin-α3, importin-α4, importin-α5, importin-α6, importin-β1, importin-β2, a nucleoporin, a Ran protein, or transportin. In some embodiments, the inhibitor of the importin pathway inhibits expression of one or more of IPO4, IPO5, IPO7, IPO11, IPO13, KPNA1, KPNA2, KPNA3, KPNA4, KPNA5, KPNA6, KPNB1, and TNPO1.

Compositions and methods for reducing cell penetration and/or nuclear localization of nuclear penetrating antibodies are disclosed. The methods generally include contacting cells with an effective amount of importin inhibitor (for example, ivermectin, mifepristone, or importazole for the inhibition of the importin pathway) to reduce nuclear localization of the antibodies. The contacting can occur in vitro or in vivo. In some embodiments cells are pre-treated or pre-incubated of importin inhibitor. Typically, the antibodies have a putative nuclear localization signal that facilitates their transport into the nucleus via the importin pathway.

The antibodies can be autoantibodies with intranuclear targets. The antibodies can cause one or more symptoms or pathologies of an autoimmune disease. Thus, methods of treating autoimmune diseases by administering a subject in need thereof an effective amount of an importin inhibitor are also provided. In specific embodiments, the autoimmune disease is scleroderma or a form of lupus, for example systemic lupus erythematosus.

In preferred embodiments, the inhibitor of the importin pathway is selected from the group consisting of a macrocyclic lactone, a diaminoquinazoline, a quinoxaline, a steroid, a peptide inhibitor, a peptidomimetic inhibitor, a retinoid derivative and an oligonucleotide inhibitor. In some embodiments, the inhibitor of the importin pathway is a binding protein. In some embodiments, the macrocyclic lactone such is an avermectin or a milbemycin. Exemplary avermectins and milbemycins include, but are not limited to, avermectin B₁ₐ/B_{1b} (abamectin), 22,23-dihydroavermectin B₁ₐ/B_{1b} (ivermectin), doramectin, moxidectin, dimadectin, emamectin, eprinomectin, latidectin, lepimectin, selamectin, milbemycin D, milbemectin, milbemycin B, milbemycin oxime, nemadectin, and combinations thereof. In one embodiment, the diaminoquinazoline is importazole. In one embodiment, the steroid is mifepristone.

Compositions, formulations, and dosage forms including an effective amount of the inhibitors of the importin pathway to reduce nuclear localization of a nuclear penetrating antibody are also provided. The compositions can be employed in the disclosed methods.

Exemplary dosages ranges and dosage regimens are also provided.

### BRIEF DESCRIPTION OF THE DRAWINGS

FIG. 1 is a line graph showing the relative intensity of variant 13 (compared to cells treated with variant 13 in the absence of ivermectin) nuclear staining in DLD1 cells co-incubated with varying concentrations of ivermectin.

FIG. 2 is a bar graph showing quantification of relative intensity of variant 13 nuclear staining in untransfected cells with intact importin 1 (positive), cells transfected with control siRNA (Ctrl siRNA), and importin β1 knockdowns (Knockdown) after treatment with 10 μM variant 13 for 30 minutes. n=100 cells per treatment, and **** represents P≤0.001.

### DETAILED DESCRIPTION OF THE INVENTION

### I. Definitions

As used herein, the term “C₁-C₆ alkyl” when used alone or in combination with other terms, comprises a straight chain or branched C₁-C₆ alkyl which refers to monovalent alkyl groups having 1 to 6 carbon atoms.

As used herein, the term “C₂-C₆ alkenyl” when used alone or in combination with other terms, comprises a straight chain or branched C₂-C₆ alkenyl. It may have any available number of double bonds in any available positions, and the configuration of the double bond may be the (E) or (Z) configuration.

As used herein, the term “C₃-C₈-cycloalkyl” refers to a saturated carbocyclic group of from 3 to 8 carbon atoms having a single ring (e.g., cyclohexyl) or multiple condensed rings.

Unless otherwise constrained by the definition of the individual substituent, all the above substituents should be understood as being all optionally substituted.

Unless otherwise constrained by the definition of the individual substituent, the term “substituted” refers to groups substituted with from 1 to 5 substituents selected from the group consisting of “C₁-C₆ alkyl,” “C₂-C₆ alkenyl,” “C₂-C₆ alkynyl,” “C₃-C₈-cycloalkyl,” “heterocycloalkyl,” “C₁-C₆ alkyl aryl,” “C₁-C₆ alkyl heteroaryl,” “C₁-C₆ alkyl cycloalkyl,” “C₁-C₆ alkyl heterocyclo alkyl,” “amino,” “aminosulfonyl,” “ammonium,” “acyl amino,” “amino carbonyl,” “aryl,” “heteroaryl,” “sulfinyl,” “sulfonyl,” “alkoxy,” “alkoxy carbonyl,” “carbamate,” “sulfanyl,” “halogen,” trihalomethyl, cyano, hydroxy, mercapto, nitro, and the like.

The term “binding protein” is used in the context of the present disclosure to refer to human immunoglobulin molecules that bind and inhibit an importin disclosed herein and includes both polyclonal and monoclonal antibodies. The term also includes genetically engineered forms such as heteroconjugate antibodies (e.g., bispecific antibodies). The term “binding protein” also includes antigen binding forms of antibodies, including fragments with antigen-binding capability (e.g., Fab′, F(ab′)₂, Fab, Fv and rIgG as discussed in Pierce Catalogue and Handbook, 1994-1995 (Pierce Chemical Co., Rockford, Ill.); Kuby, J., Immunology, 3^{rd} Ed., W.H. Freeman & Co., New York (1998). The term is also used to refer to recombinant single chain Fv fragments (scFv) as well as divalent (di-scFv) and trivalent (tri-scFV) forms thereof. The term antibody also includes bivalent or bispecific molecules, diabodies, triabodies, and tetrabodies. Examples of bivalent and bispecific molecules are described in Kostelny et al. (1992) J Immunol 148:1547; Pack and Pluckthun (1992) Biochemistry 31:1579; Hollinger et al., 1993, supra, Gruber et al. (1994) J. Immunol.:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

As used herein, the term “single chain Fv” or “scFv” as used herein means a single chain variable fragment that includes a light chain variable region (V_{L}) and a heavy chain variable region (V_{H}) in a single polypeptide chain joined by a linker which enables the scFv to form the desired structure for antigen binding (i.e., for the V_{H} and V_{L} of the single polypeptide chain to associate with one another to form a Fv). The V_{L} and V_{H} regions may be derived from the parent antibody or may be chemically or recombinantly synthesized.

As used herein, the term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917).

As used herein, the term “Framework Region” or “FR” residues are those variable domain residues other than the hypervariable region residues as herein defined.

As used herein, the term “antibody” refers to natural or synthetic antibodies that bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that bind the target antigen.

As used herein, the term “nuclear penetrating antibody” refers to an antibody, or antigen binding fragment or molecule thereof that is transported into the nucleus of living mammalian cells and binds to a target therein (e.g., a nuclear localized ligand). Exemplary targets include, but are not limited proteins and nucleic acids. An antibody that binds to DNA (e.g., single-stranded and/or double-stranded DNA) can be referred to as an anti-DNA antibody. In some embodiments, a nuclear penetrating antibody is transported into the nucleus of a cell without the aid of a carrier or conjugate. In another embodiment, a nuclear penetrating antibody is conjugated to a cell and/or nuclear-penetrating moiety, such as a cell penetrating peptide. One of skill in the art will appreciate that the term “nuclear penetrating” can be used in the context of the present disclosure to refer to other particles having a targeting moiety that targets a nuclear ligand such as scFv. For example, the term can be used to refer to a scFv that is transported into the nucleus of a cell without the aid of a carrier or conjugate and binds a nuclear ligand (e.g., single-stranded and/or double-stranded DNA, RNA, protein, etc.).

As used herein, the term “specifically binds” refers to the binding of an antibody to its cognate antigen (for example DNA) while not significantly binding to other antigens. Preferably, an antibody “specifically binds” to an antigen with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with that second molecule.

As used herein, the term “monoclonal antibody” or “MAb” refers to an antibody obtained from a substantially homogeneous population of antibodies, i.e., the individual antibodies within the population are identical except for possible naturally occurring mutations that may be present in a small subset of the antibody molecules.

One of skill in the art will appreciate that the “importin pathway” transports protein molecules into the nucleus by binding to specific recognition sequences, called nuclear localization sequences (NLS). Importin has two general subunits, importin α and importin β which represent and/or interact with a larger family of proteins comprising importin-4, importin-5, importin-7, importin-8, importin-9, importin-11, importin-13, importin-α1, importin-α2, importin-α3, importin-α4, importin-5, importin-α6 and importin-β1, importin-β2, a nucleoporin, a Ran protein. Accordingly, in an example, an inhibitor of the importin pathway inhibits one or both of importin a and importin. In another example, an inhibitor of the importin inhibits one or more of importin-4, importin-5, importin-7, importin-8, importin-9, importin-11, importin-13, importin-α1, importin-α2, importin-α3, importin-α4, importin-α5, importin-α6 and importin-β1, importin-β2, a nucleoporin, a Ran protein. Thus an inhibitor of the importin pathway can inhibit a structure or function of the nuclear pore. In another example, an importin inhibitor inhibits expression of a gene encoding an above referenced importin or other importin pathway or nuclear pore protein. For example, an importin inhibitor can inhibit expression of one or more of IPO4, IPO5, IPO7, IPO11, IPO13, KPNA1, KPNA2, KPNA3, KPNA4, KPNA5, KPNA6, KPNB1 and TNPO1.

As used herein, the term “DNA repair” refers to a collection of processes by which a cell identifies and corrects damage to DNA molecules. Single-strand defects are repaired by base excision repair (BER), nucleotide excision repair (NER), or mismatch repair (MMR). Double-strand breaks are repaired by non-homologous end joining (NHEJ), microhomology-mediated end joining (MMEJ), or homologous recombination. After DNA damage, cell cycle checkpoints are activated, which pause the cell cycle to give the cell time to repair the damage before continuing to divide. Checkpoint mediator proteins include BRCA1, MDC1, 53BP1, p53, ATM, ATR, CHK1, CHK2, and p21.

As used herein, the term “impaired DNA repair” refers to a state in which a mutated cell or a cell with altered gene expression is incapable of DNA repair or has reduced activity or efficiency of one or more DNA repair pathways or takes longer to repair damage to its DNA as compared to a wild type cell.

As used herein, the term “tumor” or “neoplasm” refers to an abnormal mass of tissue containing neoplastic cells. Neoplasms and tumors may be benign, premalignant, or malignant.

As used herein, the term “cancer” or “malignant neoplasm” refers to a cell that displays uncontrolled growth and division, invasion of adjacent tissues, and often metastasizes to other locations of the body.

As used herein, the term “inhibit” means to decrease an activity, response, condition, disease, or other biological parameter. This can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, the term “fusion protein” refers to a polypeptide formed by the joining of two or more polypeptides through a peptide bond formed between the amino terminus of one polypeptide and the carboxyl terminus of another polypeptide or through linking of one polypeptide to another through reactions between amino acid side chains (for example disulfide bonds between cysteine residues on each polypeptide). The fusion protein can be formed by the chemical coupling of the constituent polypeptides or it can be expressed as a single polypeptide from a nucleic acid sequence encoding the single contiguous fusion protein. Fusion proteins can be prepared using conventional techniques in molecular biology to join the two genes in frame into a single nucleic acid sequence, and then expressing the nucleic acid in an appropriate host cell under conditions in which the fusion protein is produced.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

As used herein, the term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or includes a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, the term “sustained release” refers to release of a substance over an extended period of time in contrast to a bolus type administration in which the entire amount of the substance is made biologically available at one time.

As used herein, the phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other adverse events, commensurate with a reasonable benefit/risk ratio.

As used herein, the phrase “pharmaceutically acceptable carrier” refers to pharmaceutically acceptable materials, compositions or vehicles, such as a liquid or solid filler, diluent, stabilizers, solvent or encapsulating matrix involved in carrying or transporting any subject composition, from one organ, or portion of the body, to another organ, or portion of the body. Each carrier must be “acceptable” in the sense of being compatible with the other ingredients of a subject composition and not injurious to the patient.

As used herein, the phrase “pharmaceutically acceptable salts” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts.

As used herein, the term “individual,” “host,” “subject,” and “patient” are used interchangeably to refer to any individual who is the target of administration or treatment. The subject can be a vertebrate, for example, a mammal. Thus, the subject can be a human or veterinary patient. In an example, the subject has an autoimmune disease such as lupus.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, “active agent” refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, weight, etc.), the disease or disorder being treated, as well as the route of administration, the pharmacokinetics of the agent being administered and the pharmacodynamic effects of the active.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

The term “subject” or “patient” refers to any individual who is the target of administration. The subject can be a vertebrate, for example, a mammal. Thus, the subject can be a human. The subject can be domesticated, agricultural, or zoo- or circus-maintained animals. Domesticated animals include, for example, dogs, cats, rabbits, ferrets, guinea pigs, hamsters, pigs, monkeys or other primates, and gerbils. Agricultural animals include, for example, horses, mules, donkeys, burros, cattle, cows, pigs, sheep, and alligators. Zoo- or circus-maintained animals include, for example, lions, tigers, bears, camels, giraffes, hippopotamuses, and rhinoceroses. The term does not denote a particular age or sex.

### II. Compositions

The disclosed compositions typically are, or include, one or more importin pathway inhibitors. In some embodiments, the inhibitor of the importin pathway is selected from the group consisting of a macrocyclic lactone, a diaminoquinazoline, a quinoxaline, a steroid, a peptide inhibitor, a peptidomimetic inhibitor, a retinoid derivative and an oligonucleotide inhibitor.

A. Macrocyclic Lactones

In some embodiments, the one or more importin pathway inhibitors is a macrocyclic lactone or combination of two or more macrocyclic lactones, for example one or more of the macrocyclic lactones disclosed herein or tautomers, geometrical isomers, optically active forms, enantiomeric mixtures thereof, pharmaceutically acceptable salts or pharmaceutically active derivatives thereof. Exemplary macrocyclic lactones are described in, for example, U.S. Published Application Nos. 2014/0080779 and 2002/0160967, Macrocyclic Lactones in Antiparasitic Therapy, ed. J. Vercruysse and R. S. Rew, CAB International North America, 2002, and discussed in more detail below.

Macrocyclic lactones include fermentation products, and chemical derivatives thereof, of microorganisms, in particular soil microorganisms, such as those belonging to the genus Streptomyces. In particular, macrocyclic lactones include fermentation products, and chemical derivatives thereof, produced for example, by Streptomyces avermitilis, also called avermectins and produced for example, by Streptomyces hygroscopicus, also called milbemycins.

In particular embodiments, the one or more macrocyclic lactones is selected from avermectin B₁ₐ/B_{1b} (abamectin), 22,23-dihydroavermectin B₁ₐ/B_{1b} (ivermectin), doramectin, moxidectin, dimadectin, emamectin, eprinomectin, latidectin, lepimectin, selamectin, milbemycin D, milbemectin, milbemycin B, milbemycin oxime, nemadectin, and combinations thereof.

For example, the importin pathway inhibitor can be a macrocyclic lactone of Formula (I):

[chemical structure]

as well as tautomers, geometrical isomers, optically active forms, enantiomeric mixtures thereof, pharmaceutically acceptable salts and pharmaceutically active derivative thereof, wherein —X— is selected from —CH═CH, —CH₂—CH(OH)—, —(CH₂)₂—, and —CH₂—C(═N—OCH₃)—, —Y— is selected from —CH(OR₄)—, wherein R₄ is selected from hydrogen and methyl; —C(═N—OH)—; and —CH(OCH₃)—; R₃ is selected from optionally substituted C₁-C₆ alkyl such as sec-butyl, iso-butyl, optionally substituted propyl (e.g. isopropyl, methyl-1 propyl) and optionally substituted C₂-C₆ alkenyl such as optionally substituted hexenyl (e.g. —C(CH₃)═CH—CH(CH₃)₂) and optionally substituted C₃-C₈-cycloalkyl such as optionally substituted cyclohexyl (e.g. cyclohexyl), and R₅ is selected from H and a group of Formula (II):

[chemical structure]

wherein R¹ is selected from —OH, —NH—C(O)—CH₃ and —NH—CH₃ and n is an integer selected from 0 and 1.

In another example, the macrocyclic lactone is a macrocyclic lactone of Formula (I) or tautomer, geometrical isomer, optically active form, enantiomeric mixture thereof, pharmaceutically acceptable salt and pharmaceutically active derivative thereof, wherein —X— is —CH═CH—, —Y— is —CH(OH)—, —R₃— is optionally substituted alkyl; R₅ is a group of Formula (II) wherein R¹ is OH and n is 1.

In another example, the macrocyclic lactone is a macrocyclic lactone of Formula (I) or tautomer, geometrical isomer, optically active form, enantiomeric mixture thereof, pharmaceutically acceptable salt and pharmaceutically active derivative thereof, wherein —X— is —CH═CH—, —Y— is —CH(OH)—, —R₃— is optionally substituted C₃-C₈-cycloalkyl; R₅ is a group of Formula (II) wherein R¹ is OH and n is 1.

In another example, the macrocyclic lactone is a macrocyclic lactone of Formula (I) or tautomer, geometrical isomer, optically active form, enantiomeric mixture thereof, pharmaceutically acceptable salt and pharmaceutically active derivative thereof, wherein —X— is —CH₂—CH₂—, —Y— is —CH(OH)—, —R₃— is optionally substituted alkyl; R₅ is a group of Formula (II) wherein R¹ is OH and n is 1.

In another example, the macrocyclic lactone is a macrocyclic lactone of Formula (I) or tautomer, geometrical isomer, optically active form, enantiomeric mixture thereof, pharmaceutically acceptable salt and pharmaceutically active derivative thereof, wherein R₅ is H and X, Y, R₃, R¹ and n are as described above.

1. Avermectins

In some embodiments, the macrocyclic lactone is an avermectin, or a mixture thereof.

Avermectins can be isolated by standard methods known in the art, for example as described in U.S. Pat. No. 4,160,084; Albers-Schonberg et al., 1981, above; or by genetic engineering of microorganisms as described in U.S. Pat. No. 5,252,474 or by synthetic methods described in Danishefsky et al., 1989, above and in Pitterna 2009, Bioorganic & Medicinal Chemistry 17, 4085-4095. The term avermectins refers to compounds that are described in Albers-Schonberg et al., 1981, above; Danishefsky et al., 1989, J. Am. Chem. Soc., 111, 2967-2980; Burg et al., 1979; Lankas et al., 1989, Toxicology. In Ivermectin and Abamectin, Campbell, W. C., Ed. Springer Verlag, New York, N.Y., 1989, 10-142; U.S. Pat. No. 4,199,59; US 2009/0281175) and derivatives or mixtures thereof. Avermectins include ivermectin, abamectin, doramectin, eprinomectin, and selamectin.

Avermectins were initially isolated from the microorganism Streptomyces avermitilis as microbial metabolites (U.S. Pat. No. 4,310,519) and can occur essentially as a mixture consisting of the eight components A₁ₐ, A_{1b}, A₂ₐ, A₂, B₁ₐ, B_{1b}, B₂ₐ and B_{2b} (I. Putter et al., Experentia 37 (1981) p. 963, Birkhauser Verlag (Switzerland)). In addition the synthetic derivatives, in particular 22,23-dihydroavermectin B1 (ivermectin), are also provided (U.S. Pat. No. 4,199,569).

The use of the avermectins, e.g. 22.23-dihydroavermectins B₁ (ivermectin) and milbemycins as endoparasiticides is the subject of numerous patent applications and review articles (e.g. biological actions in: “Ivermectin and Abamectin”, W. C. Campbell, Ed., Springer Verlag, New York, N.Y., 1989; “Avermectins and Milbemycins Part II” H. G. Davies et al., Chem. Soc. Rev. 20 (1991) pp. 271-339; chemical modifications in: G. Lukacs et al. (Eds.), Springer Verlag, New York, (1990), Chapter 3; Cydectin® [moxidectin and derivatives]: G. T. Carter et al., J. Chem. Soc. Chem. Commun. (1987), pp. 402-404); EP 423 445-A1) “Doramectin—a potent novel endectocide” A. C. Goudie et al., Vet. Parasitol. 49 (1993). pp. 5-15).

Avermectins and their derivatives which may be particularly emphasized are those of the general formula (III)

[chemical structure]

wherein, R₅ is

[chemical structure]

X, R₃, and R₄ are as defined in Table 1.

TABLE 1
Avermectins of Formula III^{a}

| Macrocyclic lactone | X | R₃ | R₄ |
|---|---|---|---|
| Avermectin A₁ₐ | —CH═CH— | -sec-Bu | —Me |
| Avermectin A_{1b} | —CH═CH— | -iso-Pr | —Me |
| Avermectin A₂ₐ | —CH₂—CHOH— | -sec-Bu | —Me |
| Avermectin A_{2b} | —CH₂—CHOH— | -iso-Bu | —Me |
| Avermectin B₁ₐ | —CH═CH— | -sec-Bu | —H |
| Avermectin B_{1b} | —CH═CH— | -iso-Pr | —H |
| Avermectin B₂ₐ | —CH₂—CHOH— | -sec-Bu | —H |
| Avermectin B_{2b} | —CH₂—CHOH— | -iso-Pr | —H |
| 22.23-dihhydroavermectin B₁ₐ | —CH₂—CH₂— | -sec-Bu | —H |
| 22.23-dihhydroavermectin B_{1b} | —CH₂—CH₂— | -iso-Pr | —H |
| Doramectin | —CH═CH— | -cyclohexyl | —H |

^{a}R₅ is Formula IV in Formula III;
22.23-dihhydroavermectin B₁ is ivermectin;
sec-Bu = secondary butyl;
iso-Pr = isopropyl;
Me = methyl

The compounds of the macrocyclic lactones marked with “b” which in R₃ is an iso-propyl radical, do not necessarily have to be separated from the “a” compounds, in which R₃ is a sec-butyl group. Generally a mixture of both substances, made of >80% sec-butyl derivative (B₁ₐ) and <20% iso-propyl derivative (B_{1b}), is isolated, and can be used in the disclosed compositions and methods. Additionally, in the stereoisomers, R₅ and the substituents on a chiral carbon atom of X, can be arranged on the ring system both in the α- and β- positions, i.e., relocated above or below the plane of the molecule.

Ivermectin refers to a mixture of 22,23-Dihydroxy-Avermectin B1a and 22,23-Dihydroxy-Avermectin B1b. For example, ivermectin can be a mixture of macrocyclic lactones having at least 90% of 22,23-Dihydroxy-Avermectin B1a and about or less than 10% of 22,23-Dihydroxy-Avermectin B1b. 22,23-Dihydroxy-Avermectin B1a is also named (10E,14E,16E)-(1R,4S,5′S,6R,6′R,8R,12S,13S,20R, 21R,24S)-6′-[(S)-sec-butyl]-21,24-dihydroxy-5′,11,13,22-tetramethyl-2-oxo-(3,7,19-trioxatetracyclo[15.6.1.1^{4,8}.0^{20,24}]pentacosa-10,14,16,-22-tetraene)-6-spiro-2′-(tetrahydropyran)-12-yl 2,6-dideoxy-4-O-(2,6-dideoxy-3-O-methyl-α-L-arabino-hexopyranosyl)-3-O-methyl-α-L-arabino-hexopyrano side. 22,23-Dihydroxy-Avermectin B1b is also named (10E,14E,16E)-(1R,4S,5′S,6R,6′R,8R,12S, 13S,20R,21R,24S)-21,24-dihydroxy-6′-isopropyl-5′,11,13,22-tetramethyl-2-o-xo-(3,7,19-trioxatetracyclo[15.6.1.1^{4,8}.0^{20,24}]pentacosa-10,14,1-6,22-tetraene)-6-spiro-2′-(tetrahydro pyran)-12-yl 2,6-dideoxy-4-O-(2,6-dideoxy-3-O-methyl-α-L-arabino-hexopyranosyl)-3-O-methyl-α-L-arabino-hexo pyranoside. Ivermectin can also be used as the generic terminology for a commercial compound commercialized under the names of STROMECTOL™ (Merck & Co., Inc.) and MECTIZAN™.

Doramectin is shown in Table 1. Doramectin can also be used as the generic terminology for a commercial compound commercialized under the name of DECTOMAX™ (Pfizer).

The avermectins and 22,23-dihydroavermectins B₁(ivermectin) can be employed as mixtures. For example, abamectin contains the avermectins B₁, and their hydrogenation products, the 22,23-dihydroavermectins B₁ (ivermectin). Thus abamectin refers to a mixture of avermectin B1a and avermectin B1b. For example, abamectin can be a mixture of macrocyclic lactones having at least 80% of avermectin B1a and about or less than 20% of avermectin B1b. Abamectin can also be used as the generic terminology for a commercial compound commercialized under the names of AFFIRM™, AVID™ (Syngenta), and ZEPHYL™.

In exemplary embodiments, the one or more macrocyclic lactones includes one or more avermectins selected from abamectin, avermectin B1a, avermectin B1b, doramectin, and ivermectin. In one preferred embodiment, the one or more macrocyclic lactones is ivermectin, wherein the ivermectin is ivermectin B1a or ivermectin B1b, or a mixture thereof, which have the following structures:

[chemical structure]

2. Milbemycins

In another exemplary embodiment, the one or more macrocyclic lactones includes one or more milbemycins. Milbemycins refers to compounds including those described in Takigushi et al., 1980, J. Antibiotics, 33, 1120-1127; Mishima et al., 1974, above; Mishima et al., 1975, above; Okazaki et al., 1983, above and Takigushi et al., 1983, The Journal of Antibiotics, XXXVI (5), 502-508; U.S. Pat. No. 4,144,352 and derivatives or mixtures thereof. In particular, milbemycins includes milbemectin, milbemycin B or moxidectin, milbemycin D, Nemadectin and milbemycin oxime. Milbemycins have the same macrolide ring structure as the avermectins or 22,23-dihydroavermectins B1 (ivermectin), but carry no substituents (i.e., missing oleandrose disaccharide fragment) in position 13 (R₅=hydrogen).

As examples of milbemycins from the class of macrocyclic lactones, the compounds can have the general Formula III

[chemical structure]

in which R₅ is hydrogen, and X, R₃, and R₄ have the meaning indicated in Table 2 which follows:

TABLE 2
Examples of milbemycins having the general structure of Formula
III^{a}

| Macrocyclic | | | |
|---|---|---|---|
| lactone | X | R₃ | R₄ |
| Milbemycin | —(CH₂)₂— | iso-Pr | —H |
| B41 D | | | |
| Nemadectin | —CH₂—CHOH— | [chemical structure] | —H |
| Moxidectin | —CH₂—C(═N—OMe)— | [chemical structure] | —H |

^{a}R₅ is hydrogen in Formula III; iso-Pr = isopropyl

Milbemycins can be isolated by standard methods known in the art, for example as described in Takigushi et al., 1983, above or by synthetic methods described in Davies et al., 1986, Nat. Prod. Rep., 87. For example, milbemycin B-41 D was isolated from Streptomyces hygroscopicus by fermentation (cf. “Milbemycin: Discovery and Development”, I. Junya et al., Annu. Rep. Sankyo Res. Lab. 45 (1993), pp. 1-98; JP Pat. 8 378 549; GB 1 390 336).

B. Diaminoquinazolines

In some embodiments, the one or more importin pathway inhibitors is a diaminoquinazoline, for example one or more of the diaminoquinazolines disclosed herein or tautomers, geometrical isomers, optically active forms, enantiomeric mixtures thereof, pharmaceutically acceptable salts or pharmaceutically active derivatives thereof. In some embodiments, the diaminoquinazoline is a 2,4-diaminoquinazoline compound which has the structure of Formula V, as shown below:

[chemical structure]

wherein in the context of diaminoquinazolines, R₁, R₂, R₃, R₄, R₅, R₆, R₇, R₈, R₉ and R₁₀ may be each independently selected from H, a halogen (e.g., F, Br, Cl, or I), a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group.

In some embodiments, the diaminoquinazoline compound has the structure of Formula Va, as shown below:

[chemical structure]

wherein in the context of diaminoquinazolines, R₅ and R₆ are each independently selected from H, a halogen (e.g., F, Br, Cl, or I), a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; and

R₁₁, R₁₂ and R₁₃ are each attached to their respective rings at any available position on the ring and are each independently selected from H, a halogen (e.g., F, Br, Cl, or I), a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group.

In some embodiments, the diaminoquinazoline compound has the structure of Formula Vb, as shown below:

[chemical structure]

wherein in the context of diaminoquinazolines, R₅ is selected from H, a halogen (e.g., F, Br, Cl, or I), a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group.

In one embodiment, the diaminoquinazoline which inhibits the importin pathway is importazole (N-(1-phenylethyl)-2-pyrrolidin-1-ylquinazolin-4-amine, CAS Number 662163-81-7) which has the following structure:

[chemical structure]

C. Quinoxalines

In some embodiments, the one or more importin pathway inhibitors is a quinoxaline, for example one or more of the quinoxalines disclosed herein or tautomers, geometrical isomers, optically active forms, enantiomeric mixtures thereof, pharmaceutically acceptable salts or pharmaceutically active derivatives thereof.

In some embodiments, the quinoxaline has the following Formula VI:

[chemical structure]

wherein in the context of quinoxalines, R₁ is a C₂-C₅ alkyl group, branched or unbranched, optionally functionalized with a substituent selected from the group consisting of an amine, an imidazole, an alcohol or a morpholine; and wherein R₂ is a hydrogen or a methyl group. In some embodiments, in the context of quinoxalines, R₁ may be selected from

[chemical structure]

an ethyl group, a propyl group, a butyl group, an iso-butyl group, and iso-pentyl group, a propanol group and

[chemical structure]

wherein * represents the site of bonding to the quinoxaline.

In some embodiments, the one or more quinoxalines are selected from the following compounds:

[chemical structure]

[chemical structure]

[chemical structure]

In another embodiments, the quinoxaline is INI-43 (3-(1H-benzimidazol-2-yl)-1-(3-dimethylaminopropyl)pyrrolo[5,4-b]quinoxalin-2-amine), which has the following structure:

[chemical structure]

D. Steroids

In some embodiments, the one or more importin pathway inhibitors is a steroid, for example one or more of the steroids disclosed herein or tautomers, geometrical isomers, optically active forms, enantiomeric mixtures thereof, pharmaceutically acceptable salts or pharmaceutically active derivatives thereof.

In one embodiment, the steroid which inhibits the importin pathway is mifepristone (11β-(4-Dimethylamino)phenyl-170-hydroxy-17-(1-propynyl)-estra-4,9-dien-3-one, CAS Number 84371-65-3), which has the following structure:

[chemical structure]

E. Peptide Inhibitors

In some embodiments, the one or more importin pathway inhibitors is a peptide inhibitor, for example one or more of the peptide inhibitors disclosed herein or a derivative thereof. In some embodiments, the peptide inhibitor include the amino acid sequence RRRRPRKRPLEWDEDEEPPRKRKRLW (SEQ ID NO:65) or RRRRRRKRKREWDDDDDPPKKRRRLD (SEQ ID NO:66). Examples of such inhibitors are discussed in Kosugi et al. Chem Biol 15(9), 940-949 (2008). In another embodiment the peptide inhibitor includes the amino acid sequence GGSYNDFGNYNNQSSNFGPMKGG NFGG-RF-EPYANPTKR (SEQ ID NO:67). Examples of such inhibitors are discussed in Cansizoglu et al. Nat Struct Mol Biol 14(5), 452-454 (2007).

F. Peptidomimetic Inhibitors

In some embodiments, the one or more importin pathway inhibitors is a peptidomimetic inhibitor, for example one or more of the peptidomimetic inhibitors disclosed herein or a derivative thereof. In some embodiments, the peptidomimetic inhibitor contains a phenyl core, wherein the phenyl core is optionally substituted with one or more groups selected from acyl and amide, wherein the acyl and amide may be further substituted. In one embodiment, the peptidomimetic inhibitor is 58H5-6, which has the following structure:

[chemical structure]

In other embodiments, the peptidomimetic inhibitor contains a pyrrolidine core, wherein the pyrrolidine core may be further optionally substituted with one or more C₁₋₆alkyl, acyl and amide, wherein the acyl and amide may be further optionally substituted.

In one embodiment, the peptidomimetic inhibitor is karyostatin 1A which has the following structure:

[chemical structure]

wherein in the context of karyostatin 1A, R is H or 4-{3-{4-[(3-Aminopropyl)-aminocarbonyl]-phenyl}-1H-indazol-1yl}-benzoic acid (AIDA).

In one embodiment, the peptidomimetic inhibitor is karyostatin 1A according to the above structure, wherein R is H. In another embodiment, the peptidomimetic inhibitor is karyostatin 1A according to the above structure, wherein R is 4-{3-{4-[(3-Aminopropyl)-aminocarbonyl]-phenyl}-1H-indazol-1yl}-benzoic acid (AIDA).

G. Retinoid Derivatives

In some embodiments, the one or more importin pathway inhibitors is a retinoid derivative, for example one or more of the retinoid derivatives disclosed herein or tautomers, geometrical isomers, optically active forms, enantiomeric mixtures thereof, pharmaceutically acceptable salts or pharmaceutically active derivatives thereof.

In one embodiment, the retinoid derivative is fenretinide (N-(4-hydroxyphenyl) retinamide, CAS Number 65646-68-6), which has the following structure:

[chemical structure]

H. Oligonucleotide Inhibitors

In some embodiments, the one or more importin pathway inhibitors is an oligonucleotide inhibitor. The oligonucleotide inhibitors can be designed to target and reduced expression or translation of one or more nucleic acids (e.g., DNA including genomic DNA, RNA including mRNA, etc.) encoding a member of the importin pathway. Subunits and proteins of the importin pathway are discussed in more detail above and include proteins that contribute to the structure and function of the nuclear pore. In particular embodiments, the reduce expression or translation of one or more nucleic acids (e.g., DNA including genomic DNA, or RNA including mRNA) encoding a protein that facilitates nuclear import, including, but not limited to, an importin and nuclear pore protein such as those mentioned above. In exemplary embodiments, the oligonucleotide inhibitor reduces expression or translation of nucleoporin 85 (nup85), importin α, importin β1, importin β2, importin 13, transportin, or the GTPase Ran.

Exemplary oligonucleotide inhibitors include isolated or synthetic antisense RNA or DNA, siRNA or siDNA, miRNA, miRNA mimics, short hairpin RNA (shRNA) or DNA (shDNA) and Chimeric Antisense DNA or RNA. The term “antisense” as used herein means a sequence of nucleotides complementary to and therefore capable of binding to a coding sequence, which may be either that of the strand of a DNA double helix that undergoes transcription, or that of a messenger RNA molecule. The terms “short hairpin RNA” or “shRNA” refer to an RNA structure having a duplex region and a loop region. The term small interfering RNA (siRNA), sometimes known as short interfering RNA or silencing RNA, is a class of double-stranded RNA molecules, 20-25 base pairs in length. A siRNA that inhibits or prevents translation to a particular protein is indicated by the protein name coupled with the term siRNA. Thus a siRNA that interferes with the translation of an importin such as IPO4 is indicated by the expression “IPO4 siRNA”. The term “microRNA” (abbreviated miRNA) is a small non-coding RNA molecule (containing about 22 nucleotides) found in plants, animals and some viruses, that functions in RNA silencing and post-transcriptional regulation of gene expression. The prefix “miR” is followed by a dash and a number, the latter often indicating order of naming. Different miRNAs with nearly identical sequences except for one or two nucleotides are annotated with an additional lower case letter. Numerous miRNAs are known in the art and can be screened for capacity to inhibit an importin disclosed herein (miRBase V.21 nomenclature; Kozomara et al. 2013; Griffiths-Jones, S. 2004).

I. Binding Protein Inhibitors

In some embodiments, the one or more importin pathway inhibitors is a binding protein.

### III. Formulations

The disclosed compounds can be formulated in a pharmaceutical composition. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration.

The compositions can be administered systemically, regionally, or locally.

Drugs can be formulated for immediate release, extended release, or modified release. A delayed release dosage form is one that releases a drug (or drugs) at a time other than promptly after administration. An extended release dosage form is one that allows at least a two-fold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are prepared using a pharmaceutically acceptable carrier composed of materials that are considered safe and effective and may be administered to an individual without causing undesirable biological side effects or unwanted interactions.

The compositions can include one or more excipients. Excipients are all components present in the pharmaceutical formulation other than the active pharmaceutical agent or agent(s) (i.e., importin pathway inhibitors) being delivered. The term excipient includes, but is not limited to, diluents, binders, lubricants, disintegrants, fillers, and coating compositions. Excipient also includes all components of the coating composition which may include plasticizers, pigments, solubilizes, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et. al., (Media, PA: Williams and Wilkins, 1995) which provides information on excipients, materials, equipment and process for preparing tablets and capsules and delayed release dosage forms of tablets, capsules, and granules. See also, Handbook Of Pharmaceutical Excipients, sixth edition, Ed. By Raymond, et al., (2009).

The compound can be administered to a subject with or without the aid of a delivery vehicle. Appropriate delivery vehicles for the compounds are known in the art and can be selected to suit the particular active agent. For example, in some embodiments, the active agent(s) and/or other pharmaceutical ingredient(s) is/are incorporated into or encapsulated by, conjugated to, or otherwise bound to, a nanoparticle, microparticle, micelle, polymeric micelle, polymersome, microbubble, liposome, synthetic lipoprotein particle, dendrimer, or carbon nanotube. For example, the compositions can be incorporated into a vehicle such as polymeric particles or conjugated to dendrimer(s) which provide controlled release of the active agent(s). In some embodiments, release of the drug(s) is controlled by diffusion of the active agent(s) out of the microparticles and/or degradation or erosion of the polymeric particles by hydrolysis, osmotic release, and/or enzymatic degradation. In some embodiments, composition is administered as in situ gel forming depot that releases the active agent.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly (ester anhydrides), polycaprolactones, polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

A. Formulations for Parenteral Administration

Compounds and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants, excipients, and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, medium chain triglycerides (MCT), gelatin, and injectable organic esters such as ethyl oleate. The formulations may be lyophilized and reconstituted/resuspended immediately before use. The formulation may be sterilized by, for example, filtration through a bacteria retaining filter, by incorporating sterilizing agents into the compositions, or by irradiating the compositions.

B. Oral Immediate Release Formulations

Suitable oral dosage forms include tablets, capsules, solutions, suspensions, syrups, wafers, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

C. Extended Release Dosage Forms

The extended release formulations can be prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

D. Delayed Release Dosage Forms

Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT®. (Rohm Pharma; Westerstadt, Germany), including EUDRAGIT®. L30D-55 and L100-55 (soluble at pH 5.5 and above), EUDRAGIT®. L-100 (soluble at pH 6.0 and above), EUDRAGIT®. S (soluble at pH 7.0 and above, as a result of a higher degree of esterification), and EUDRAGITS. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Methods of Manufacturing

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, PA: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

E. Formulations for Mucosal and Pulmonary Administration

Active agent(s) and compositions thereof can be formulated for pulmonary or mucosal administration. The administration can include delivery of the composition to the lungs, nasal, oral (sublingual, buccal), vaginal, or rectal mucosa. In a particular embodiment, the composition is formulated for and delivered to the subject sublingually.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, Calif.).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different active agents may be administered to target different regions of the lung in one administration.

F. Topical and Transdermal Formulations

Transdermal formulations may also be prepared. These will typically be gels, ointments, lotions, sprays, or patches, all of which can be prepared using standard technology. In some embodiments additional or alternative formulations are administered topically or transdermally using microneedles. Transdermal formulations can include penetration enhancers.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid dispersed as small droplets within a continuous (bulk) phase. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying drug delivery systems (SEDDS). These drug delivery systems are typically capsules (hard shell or soft shell) comprised of the drug dispersed or dissolved in a mixture of surfactant(s) and lipophillic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components.

Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Additional agents that can be added to the formulation include penetration enhancers. In some embodiments, the penetration enhancer increases the solubility of the drug, improves transdermal delivery of the drug across the skin, in particular across the stratum corneum, or a combination thereof. Some penetration enhancers cause dermal irritation, dermal toxicity and dermal allergies. However, the more commonly used ones include urea, (carbonyldiamide), imidurea, N, N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.). Chemical penetrations and methods of increasing transdermal drug delivery are described in Inayat, et al., Tropical Journal of Pharmaceutical Research, 8(2):173-179 (2009) and Fox, et al., Molecules, 16:10507-10540 (2011). In some embodiments, the penetration enhancer is, or includes, an alcohol such ethanol, or others disclosed herein or known in the art.

Delivery of drugs by the transdermal route has been known for many years. Advantages of a transdermal drug delivery compared to other types of medication delivery such as oral, intravenous, intramuscular, etc., include avoidance of hepatic first pass metabolism, ability to discontinue administration by removal of the system, the ability to control drug delivery for a longer time than the usual gastrointestinal transit of oral dosage form, and the ability to modify the properties of the biological barrier to absorption.

Controlled release transdermal devices rely for their effect on delivery of a known flux of drug to the skin for a prolonged period of time, generally a day, several days, or a week. Two mechanisms are used to regulate the drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. Devices incorporating a reservoir will deliver a steady drug flux across the membrane as long as excess undissolved drug remains in the reservoir; matrix or monolithic devices are typically characterized by a falling drug flux with time, as the matrix layers closer to the skin are depleted of drug. Usually, reservoir patches include a porous membrane covering the reservoir of medication which can control release, while heat melting thin layers of medication embedded in the polymer matrix (e.g., the adhesive layer), can control release of drug from matrix or monolithic devices. Accordingly, the active agent can be released from a patch in a controlled fashion without necessarily being in a controlled release formulation.

Patches can include a liner which protects the patch during storage and is removed prior to use; drug or drug solution in direct contact with release liner; adhesive which serves to adhere the components of the patch together along with adhering the patch to the skin; one or more membranes, which can separate other layers, control the release of the drug from the reservoir and multi-layer patches, etc., and backing which protects the patch from the outer environment.

Common types of transdermal patches include, but are not limited to, single-layer drug-in-adhesive patches, wherein the adhesive layer contains the drug and serves to adhere the various layers of the patch together, along with the entire system to the skin, but is also responsible for the releasing of the drug; multi-layer drug-in-adhesive, wherein which is similar to a single-layer drug-in-adhesive patch, but contains multiple layers, for example, a layer for immediate release of the drug and another layer for control release of drug from the reservoir; reservoir patches wherein the drug layer is a liquid compartment containing a drug solution or suspension separated by the adhesive layer; matrix patches, wherein a drug layer of a semisolid matrix containing a drug solution or suspension which is surrounded and partially overlaid by the adhesive layer; and vapor patches, wherein an adhesive layer not only serves to adhere the various layers together but also to release vapor. Methods for making transdermal patches are described in U.S. Pat. Nos. 6,461,644, 6,676,961, 5,985,311, and 5,948,433.

### IV. Method of Use

A. Method of Treatment

The results below show that compounds that inhibit the importin pathway can reduce translocation of nuclear-penetrating antibodies from the cytosol into the nucleus. Thus, such compounds can be administered to a subject in need thereof in an effective amount to reduce translocation of nuclear-penetrating antibodies from the cytosol into the nucleus. The subject is can be, for example, an animal such as a human, dog, cat, cattle, sheep, pig, etc. Typically, translocation of nuclear-penetrating antibodies from the cytosol into the nucleus is reduced in an amount effective to reduce one or more symptoms or conditions caused by, or associated with, a nuclear-penetrating antibody or antibodies. The antibody or antibodies can be autoantibodies. The antibodies can be anti-DNA antibodies. Exemplary anti-DNA/anti-nucleosome antibodies are known in the art (see, e.g., Shuster A. M. et. al., Science, v. 256, 1992, pp. 665-667, Isenberg, et al., Rheumatology, 46 (7):1052-1056 (2007))). For example, autoantibodies to single or double stranded deoxyribonucleic acid (DNA) are frequently identified in the serum of patients with systemic lupus erythematosus (SLE) and are often implicated in disease pathogenesis.

The precise dosage will vary according to a variety of factors such as subject-dependent variables (e.g., age, immune system health, clinical symptoms etc.). Exemplary dosages, symptoms, pharmacologic, and physiologic effects are discussed in more detail below.

In some embodiments, the compounds are administered in bolus, pulsatile, delayed release, dosage escalation, or dosage de-escalation fashion.

1. Exemplary Dosages

Typically, the disclosed compositions are administered in an effective therapeutic dose that can be between, for example, 100 μg and 5 g, or between 100 μg and 3 g, between 100 μg and 1 g, or between 150 μg and 500 mg, or between 200 μg and 200 mg per day, or between 1 mg and 35 mg, or between 2 mg and 35 mg, or between 3 mg and 20 mg.

Dosages for oral and topical administration to humans of macrocyclic lactones such as ivermectin are known in the art for treating conditions such as onchocerciasis, strongyloidiasis, pediculosis, and acne rosacea (see, e.g., Moustafa, et al., Drugs, 74:1457-65 (2014), doi: 10.1007/s40265-014-0281-x.

For example a single oral dose can be between about 10 μg/kg and about 10 mg/kg, or about 25 μg/kg and about 7.5 mg/kg, or about 50 μg/kg and about 5 mg/kg, or about 50 μg/kg and about 500 μg/kg. Maximum oral dosages are generally in the range about 150-200 μg/kg single dose for most indications; up to 400 μg/kg PO for Bancroft's filariasis, and up to about 4 oz/topical application of 0.5%-1% w/v lotion.

An exemplary dosage guide for a single oral administration based on body weight is 15 to 25 kg: 3 mg; 26 to 44 kg: 6 mg; 45 to 64 kg: 9 mg; 65 to 84 kg: 12 mg; 85 kg or more: 0.15 mg/kg.

Another exemplary dosage guide for a single oral administration based on body weight is 15 to 24 kg: 3 mg; 25 to 35 kg: 6 mg; 36 to 50 kg: 9 mg; 51 to 65 kg: 12 mg; 66 to 79 kg: 15 mg; 80 kg or more: 0.2 mg/kg.

U.S. Published Application No. 2014/0080779 provides a dosage range of 30 mg/kg body weight to 120 mg/kg body weight of macrocyclic lactone for treatment of colorectal cancer.

As mentioned above, topical formulations are often prepared with about 0.5% to about 1% active agent. Injectable formulations are also known. See, for example, U.S. Published Application No. 2002/0160967, which reviews injectable formulations, and recommends 0.2 to 5% m/v of active compound, wherein 1% m/v means, for example, 10 mg of active compound in 1 ml of solution.

Dosages for oral administration to animals (e.g., dogs, cats, cattle, sheep, pigs, etc.) of macrocyclic lactones such as ivermectin are also known in the art. For example, HEARTGARD® Chewables are a flavored treatment for dogs given once a month to prevent heartworms. The recommend dosage is as follows: 25 pounds is 68 μg, for dogs 26-50 pounds is 136 μg and for dogs 51-100 pounds is 272 μg.

Other exemplary dosages are provided in the chart below, adapted from Veterinary Pharmacology and Therapeutics, Ninth Edition, Riviere and Papich, ed., John Wiley &Sons, Mar. 17, 2009, pg. 1134.

TABLE 3
Formulations of avermectin-type compounds commercially
available for use in different animal species

| | Formulation and | Dose | | Target |
|---|---|---|---|---|
| Drug | Administration Route | Rate | | Species |
| IVERMECTIN | 1% injectable formulation | 0.2 | mg/kg | Cattle, |
| (LVOMEC ® | in propylene glycol/ | 0.3 | mg/kg | sheep |
| Merial Ltd.) | glycerol formal (60:40) | 0.4 | mg/kg | Pigs |
| | (SC) | | | Goats |
| | 3.15% injectable oil-based | 0.63 | mg/kg | Cattle |
| | long-acting formulations | | | |
| | (SC) | | | |
| | 0.5% transdermal | 0.5 | mg/kg | Cattle |
| | formulation in isopropyl | | | |
| | alcohol (pour-on) | | | |
| | Sustained release bolus | 12 mg/day | | Cattle |
| | (oral) | for 135 days | | |
| | Controlled released | 1.6 mg/day | | Sheep |
| | capsules (oral) | for 100 days | | |
| | Micelar drench | 0.2 | mg/kg | Sheep |
| | formulation (oral) | 0.4 | mg/kg | Goats |
| | Liquid solution (oral)(#) | 0.2 | mg/kg | Horses |
| | 1.87% paste formulation | 0.2 | mg/kg | Horses |
| | in a titanium dioxide and | | | |
| | propylene glycol vehicle | | | |
| | (oral) (#) | | | |
| | In feed formulation | 0.1 mg/kg | | Pigs |
| | (Premix) (oral) | for 7 days | | |
| | Flavored chewable and | 0.006 | mg/kg | Dogs |
| | tablets formulation (oral) | 0.024 | mg/kg | Cats |
| | (##) | | | |
| ABAMECTIN | 1% injectable formulation | 0.2 | mg/kg | Cattle, |
| (DUOTIN ® | in propylene glycol/ | | | sheep |
| Merial Ltd.) | glycerol formal (60:40) | | | |
| | (SC) | | | |
| DORAMECTIN | 1% injectable formulation | 0.2 | mg/kg | Cattle, |
| (DECTOMAX ® | in sesame oil/ethyl locate | 0.3 | mg/kg | sheep |
| Pfizer) | (90:10)(SC, IM) | 0.5 | mg/kg | Pigs |
| | 0.5% transdermal | | | Cattle |
| | formulation (pour-on) | | | |
| EPRINOMECTIN | 0.5% transdermal | 0.5 | mg/kg | Beef |
| (EPRINEX ® | formulation (pour-on) | | | and |
| Merial Ltd.) | | | | dairy |
| | | | | cattle |
| SELAMECTIN | 6% and 12% transdermal | 6 | mg/kg | Dogs, |

| | | |
|---|---|---|
| (REVOLUTION ® | formulations in an | cats |
| Pfizer) | isopropyl/dipropylene | |
| | glycol methyl-ether | |
| | vehicle (pour-on) | |

SC: subcutaneous;
IM: intramuscular
Trade names
(#) EQVALAN ®, Meral Ltd.
(##) HEARTGARD ®, Merial Ltd.

Table 3 does not include information on different hemoctin generic preparation available in some countries, particularly for the classic 1% and long-acting formulations for use in cattle.

TABLE 4
Formulations of milbemycin-type compounds commercially
available for use in different animal species

| | Formulation and | | | Target |
|---|---|---|---|---|
| Drug | Administration Route | Dose Rate | | Species |
| MILBEMIYCIN | Flavor tablets (oral) | 0.5 | mg/kg | Dogs |
| OXIME | | 2 | mg/kg | Cats |
| (INTERCEPTOR ®, | | | | |
| Novartis) | | | | |
| MOXIDECTIN | 1% injectable | 0.2 | mg/kg | Cattle, |
| (CYDECTIN ® Fort | formulation | 0.3 | mg/kg | sheep |
| Dodge) | (SC) | | | Pigs |
| | 10% long-acting oil-based | 1 | mg/kg | Cattle |
| | formulation (SC in the | | | |
| | base of the ear) | | | |
| | 0.5% transdermal | 0.5 | mg/kg | Beef |
| | formulation (pour-on) | | | and |
| | | | | dairy |
| | | | | cattle |
| | Drench formulation (oral) | 0.2 | mg/kg | Sheep |
| | 2% long-acting oil-based | 1 | mg/kg | Sheep |
| | formulation (SC in the | | | |
| | base of the ear) | | | |
| | Gel formulation (oral) (#) | 0.4 | mg/kg | Horses |
| | Tablets (oral) (##) | 0.003 | mg/kg | Dogs |
| | Sustained release | 0.17 | mg/kg | Dogs |

injectable formulation
(SC) (###)

SC: subcutaneous;
IM: intramuscular
Trade names
(#) EQVALAN ®, Fort Dodge
(##) PROHEART ®, Fort Dodge
(###) PROHEART 6 ®, Fort Dodge

In some embodiments, the disclosed method includes administration of similar dosages to those known for treating other diseases and conditions such as those discussed above. In some embodiments, the dosages are different.

2. Exemplary Treatment Schedules

Dosage regimens are also provided. In some embodiments, the composition is administered to the subject two or more times in intervals of hours, days, or weeks. In some embodiments, the composition is administered to a subject in need thereof once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments the composition is administered to a subject one, twice, or three times weekly. In some embodiments, the composition is administered to a subject one, twice, or three times monthly. In some embodiments, the compound is administered to a subject in need thereof according to the CDC's recommended ivermectin dosage schedule for treating crusted scabies: 200 μg/kg orally with food) is given as five doses (approximately days 1, 2, 8, 9, and 15), with consideration of two additional doses (approximately days 22 and 29) for severe cases. Re-treatment 2 weeks after the initial treatment regimen can be considered for those persons who are still symptomatic.

In some embodiments, a single dose is sufficient to improve one or more symptoms of a disease. In some embodiments two or more doses are needed. In some embodiments, the subject is administered according to a dosage regimen that ends, for example after two weeks. In some embodiments, the dosage regimen is repeated indefinitely. In some embodiments, single treatments or entire treatment regimens can be repeated 1, 2, 3, 4, 5, 6, 7, or more days, weeks, or months apart.

3. Combination Therapy

In some embodiments, the importin inhibitor is administered in combination with one or more additional active agents. The combination therapies can include administration of the active agents together in the same admixture, or in separate admixtures. Therefore, in some embodiments, the pharmaceutical composition includes two, three, or more active agents. Such formulations typically include an effective amount of importin inhibitor. The different active agents can have the same or different mechanisms of action. In some embodiments, the combination results in an additive effect on the treatment of the disease or disorder. In some embodiments, the combinations results in a more than additive effect on the treatment of the disease or disorder.

The pharmaceutical compositions can be formulated as a pharmaceutical dosage unit, also referred to as a unit dosage form, which can include a single effective dose of importin inhibitor. Exemplary combination therapies are discussed in more detail below.

B. Diseases and Disorders to be Treated

Nuclear penetrating antibodies are believed to play a role in various autoimmune disorders such as systemic lupus erythematosus and scleroderma (e.g. Mok and Lau J Clin Pathol. 56:481-490 (2003); DeFranco, Immunol Cell Biol., 94(10): 918-924 (2016); Silosi, et al., Rom J Morphol Embryol, 57(2 Suppl):633-638 (2016)). The experiments discussed in more detail below indicate that inhibition of the importin pathway may provide an effective means for treating these disorders. Thus, the disclosed compositions and methods can be used to treat autoimmune diseases, particularly autoimmune diseases that have symptoms or pathology dependent on or otherwise caused by nuclear penetrating antibodies, for example, nuclear penetrating autoantibodies.

Autoantibodies are responsible for disease manifestations in a variety of autoimmune diseases, including, systemic lupus erythematosus (lupus or SLE), systemic sclerosis (scleroderma), Graves' disease, myastenia gravis, autoimmune hemolytic anemia, and pemphigus vulgaris, and additionally may contribute to the severity of disease in other autoimmune diseases such as rheumatoid arthritis (DeFranco, Immunol Cell Biol., 94(10): 918-924 (2016)). Other diseases with an autoimmune component include, but are not limited to, primary biliary cirrhosis, primary sclerosing cholangitis, psoriasis, psoriatic arthritis, POEMS syndrome, dermatomyositis, inclusion body myositis, inflammatory myopathies, vasculitis syndromes including but not limited to Churg-Strauss Syndrome, Wegener granulomatosis, Behcet's disease, Buerger's disease, Kawasaki disease, Takayasu's arteritis, Henoch-Schonlein purpura, Giant cell arteritis, and polyarteritis nodosa.

Autoimmune diseases can be mediated principally by autoantibodies or a combination of autoantibodies and T lymphocytes (i.e., non-principal diseases), and can be organ-specific or systemic (Silosi, et al., Rom J Morphol Embryol, 57(2 Suppl):633-638 (2016)). Thus, in some embodiments, the compositions and methods are used to treat a principal organ-specific autoimmune disease, a principal specific autoimmune disease, a non-principal organ-specific autoimmune disease, or a non-principal specific autoimmune disease.

Exemplary principal organ-specific autoimmune diseases include, but are not limited to, autoimmune hemolytic anemia, autoimmune thrombocytopenia, autoimmune atrophic gastritis of pernicious anemia, myastenia gravis; and Goodpasture's syndrome.

Exemplary principal systemic disease autoimmune diseases include, but are not limited to, systemic lupus erythematosus (lupus or SLE).

In some embodiments, the subject has nephritis and the method reduces the nephritis, or prevents advancement of the nephritis. Nephritis is inflammation of the kidneys and may involve the glomeruli, tubules, or interstitial tissue surrounding the glomeruli and tubules. Nephritis is often caused by infections, and toxins, but is often caused by autoimmune disorders, such as SLE, that affect the major organs like kidneys. In some embodiments, the subject has lupus nephritis. Preferably the method improves kidney function, particularly in subjects with lupus.

Exemplary non-principal organ-specific disease autoimmune diseases involving both T lymphocytes and antibodies include, but are not limited to, diabetes mellitus, multiple sclerosis, Hashimoto's thyroiditis, and Crohn's disease.

Exemplary non-principal systemic disease autoimmune diseases involving both T lymphocytes and antibodies include, but are not limited to, rheumatoid arthritis, systemic sclerosis, and Sjögren's syndrome.

In organ-specific autoimmune diseases (such as myasthenia gravis or pemphigus), autoantibodies directly bind to and injure target organs. In some diseases, the autoimmune aggression results in the complete and irreversible loss of function of the targeted tissue (e.g., Hashimoto's thyroiditis or insulin-dependent diabetes). The autoimmune reactions may cause persistent lesions inducing an overstimulation or inhibition of its function (e.g., Graves-Basedow disease or myasthenia gravis). In other autoimmune conditions, the pathogenic events are multiple and produce destruction of several tissues (e.g., SLE).

In some embodiments, the subject does not have one or more of onchocerciasis, strongyloidiasis, pediculosis, rosacea, Bancroft's filariasis, scababies, colorectal cancer, or an infestation of an ectoparasite such as the Demodex mite.

1. Lupus

In some embodiments, the compositions and methods are used to treat a type or form of lupus.

Lupus is a chronic inflammatory disease that can affect many different parts of the body, and can cause damage to, for example, the skin, joints, kidneys, lungs, blood cells, heart, and brain. People with lupus may experience periods of flares when symptoms show up, and periods of remission when symptoms are under control. During a lupus flare, symptoms such as exhaustion, weight loss, fever, and anemia often occur. Lupus can cause damage to many parts of the body, potentially leading to the following complications: kidney failure, blood problems, such as anemia (low red blood cell count), bleeding, or clotting, high blood pressure, vasculitis (inflammation of the blood vessels), memory problems, behavior changes or hallucinations, seizures, stroke, heart disease or heart attack, lung conditions, such as pleurisy (inflammation of the chest cavity lining) or pneumonia, infections, cancer, and avascular necrosis (death of bone tissue due to a lack of blood supply).

Types of lupus include, systemic lupus erythematosus, or SLE (which is the most common form of lupus), discoid lupus erythematosus (which leads to a skin rash), subacute cutaneous lupus erythematosus (which leads to skin sores on areas of the body exposed to the sun), neonatal lupus (which affects newborns), and drug-induced lupus (which can be caused by certain medicines).

The presence of circulating autoantibodies reactive against DNA (anti-DNA antibodies) is a hallmark laboratory finding in patients with systemic lupus erythematosus (SLE). Although the precise role of anti-DNA antibodies in SLE is unclear, it has been proposed that the antibodies play an active role in SLE pathophysiology. Select lupus anti-DNA autoantibodies can penetrate into live cell nuclei and inhibit DNA repair or directly damage DNA.

In some embodiments, the subject is administered a compound that inhibits the importin pathway in combination with one or more additional active agents traditionally used to treat lupus. Traditional treatment for lupus includes non-steroidal anti-inflammatory drugs (NSAIDs), corticosteroids, immunosuppressants, hydroxychloroquine, and methotrexate. Disease-modifying antirheumatic drugs (DMARDs) are used preventively to reduce the incidence of flares, the progress of the disease, and the need for steroid use. When flares occur, they can be treated with corticosteroids. DMARDs commonly in use include antimalarials such as hydroxychloroquine and immunosuppressants (e.g. methotrexate and azathioprine). In more severe cases, immune modulators (such as corticosteroids and immunosuppressants) can be used to control the disease and prevent recurrence of symptoms. Steroid usage may lead a subject to develop Cushing's syndrome, symptoms of which may include obesity, puffy round face, diabetes mellitus, increased appetite, difficulty sleeping and osteoporosis. Subjects can also experience chronic pain, leading to administration of prescription analgesics including opioids if over-the-counter NSAIDs are insufficient. Intravenous immunoglobulins can be used to control SLE with organ involvement, or vasculitis. It is believed that they reduce antibody production or promote the clearance of immune complexes from the body, even though their mechanism of action is not well understood.

Having lupus can increase an individual's risk for cancer. Thus, in some embodiments, the subject has both an autoimmune disease such as lupus and a cancer. In some embodiments, the subject is administered a compound that inhibits the importin pathway in combination with one or more anti-cancer agents. Additional therapeutic agents include conventional cancer therapeutics such as chemotherapeutic agents, cytokines, chemokines, and radiation therapy. The majority of chemotherapeutic drugs can be divided in to: alkylating agents, antimetabolites, anthracyclines, plant alkaloids, topoisomerase inhibitors, and other antitumor agents. All of these drugs affect cell division or DNA synthesis and function in some way. Additional therapeutics include monoclonal antibodies and the new tyrosine kinase inhibitors e.g. imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab (HERCEPTIN®), cetuximab, and rituximab (RITUXAN® or MABTHERA®), bevacizumab (AVASTIN®), nivolumab, ipilimumab, pemrolizumab, immune checkpoint inhibitors, and combinations thereof.

2. Scleroderma

In some embodiments, the compositions and methods are used to treat a form or type of scleroderma.

Scleroderma is a chronic connective tissue disease generally classified as one of the autoimmune rheumatic diseases. Patients with scleroderma can have specific antibodies (ANA, anticentromere, or antitopoisomerase) in their blood that suggest autoimmunity. Symptoms can generally include thickened skin that can involve scarring, blood vessel problems, varying degrees of inflammation and pain, and is associated with an overactive immune system.

Scleroderma can be classified in terms of the degree and location of the skin and organ involvement. Accordingly, scleroderma has been categorized into two major groups, localized scleroderma and systemic sclerosis, which can be further subdivided into either diffuse or limited forms based on the location and extent of skin involvement. Localized scleroderma skin changes are in isolated areas, either as morphea patches or linear scleroderma. Morphea is scleroderma that is localized to a patchy area of the skin that becomes hardened and slightly pigmented. Sometimes morphea can cause multiple lesions in the skin. Morphea is not associated with disease elsewhere within the body, only in the involved skin areas. Linear scleroderma is scleroderma that is localized usually to a lower extremity, frequently presenting as a strip of hardening skin down the leg of a child. Linear scleroderma in children can stunt bone growth of the affected limb. Sometimes linear scleroderma is associated with a “satellite” area of a patch of localized scleroderma skin, such as on the abdomen.

The widespread type of scleroderma involves internal organs in addition to the skin. This type, called systemic sclerosis, is subcategorized by the extent of skin involvement as either diffuse or limited. The diffuse form of scleroderma (diffuse systemic sclerosis) involves symmetric thickening of skin of the extremities, face, and trunk (chest, back, abdomen, or flanks) that can rapidly progress to hardening after an early inflammatory phase. Organ disease can occur early on and be serious and significantly decrease life expectancy. Organs affected include the esophagus, bowels, and scarring (fibrosis) of the lungs, heart, and kidneys. High blood pressure can be troublesome and can lead to kidney failure (renal crisis).

The limited form of scleroderma tends to have far less skin involvement with skin thickening confined to the skin of the fingers, hands, and face. The skin changes and other features of disease tend to occur more slowly than in the diffuse form. Because characteristic clinical features can occur in patients with the limited form of scleroderma, this form has taken another name that is composed of the first initials of the common components. Thus, this form is also called the “CREST” variant (subset thereof, e.g., CRST, REST, or ST) of scleroderma. CREST syndrome represents the following features: Calcinosis (the formation of tiny deposits of calcium in the skin), Raynaud's phenomenon (the spasm of the tiny arterial vessels supplying blood to the fingers, toes, nose, tongue, or ears), Esophagus disease (characterized by poorly functioning muscle of the lower two-thirds of the esophagus), Sclerodactyly (localized thickening and tightness of the skin of the fingers or toes), and Telangiectasias (tiny red areas, frequently on the face, hands, and in the mouth behind the lips).

Some subjects have scleroderma and one or more other connective tissue diseases, such as rheumatoid arthritis, systemic lupus erythematosus, and polymyositis. Features of scleroderma along with features of polymyositis, systemic lupus erythematosus, and certain abnormal blood tests, can lead to a diagnosis of mixed connective tissue disease (MCTD).

In some embodiments, the subject is administered a compound that inhibits the importin pathway in combination with one or more additional active agents traditionally used to treat scleroderma. Current therapies use medications that focus on the four main features of the disease: inflammation, autoimmunity, vascular disease, and tissue fibrosis. Thus, subjects with scleroderma may be administered one or more anti-inflammatory agents, immunosuppressants, therapies for treating vascular disease, and/or anti-fibrotic agents. Anti-inflammatory medication include, but are not limited to, NSAIDs (e.g. ibuprofen) or corticosteroids (e.g. prednisone). Immunosuppressants include, but are not limited to, methotrexate, cyclosporine, antithymocyte globulin, mycophenolate mofetil and cyclophosphamide. Agents for treatment of vascular disease include, but are not limited to, vasodilators e.g., calcium channel blockers such as nifedipine, bosentan (endothelin-1 receptor inhibitor) and epoprostenol (prostacyclin) which can improve blood flow; agents which can reverse vasospasm such as angiotensin converting enzyme inhibitors (ACE) inhibitors, calcium channel blockers, bosentan, prostacyclin, or nitric oxide; and antiplatelet or anticoagulation therapy such as low-dose aspirin. Anti-fibrotic agents include, but are not limited to, colchicine, para-aminobenzoic acid (PABA), dimethyl sulfoxide, and D-penicillamine.

C. Methods of Modulating Anti-DNA Antibody Therapy

Select anti-DNA antibodies can penetrate into live cell nuclei and inhibit DNA repair or directly damage DNA, and efforts to use these antibodies against tumors that are sensitive to DNA damage are underway (Hansen, et al., Sci Transl Med, 4(157):157ra142 (2012), Noble, et al., Cancer Research, 2015; 75(11):2285-2291, Noble, et al., Sci Rep-Uk, 4 (2014), Noble, et al., Nat Rev Rheumatol (2016)).

A panel of hybridomas, including the 3E10 and 5C6 hybridomas was previously generated from the MRLmpj/lpr lupus mouse model and DNA binding activity was evaluated (Zack, et al., J. Immunol. 154:1987-1994 (1995); Gu, et al., J. Immunol., 161:6999-7006 (1998)). Thus in some embodiments, the anti-DNA antibody is 3E10 or 5C6 antibody or a variant, fragment, and fusion protein thereof, or a humanized form thereof.

In specific embodiments, a subject with cancer is administered a compound that inhibits the importin pathway such as ivermectin in combination with an anti-nuclear antibody, or more particularly an anti-DNA antibody such as a 3E10 or 5C6 antibody or fragment or variant or humanized form thereof. 3E10 antibodies are attracted to tumors (see, e.g., WO 2017/218825), whereas systemically administered importin pathway inhibitors such as ivermectin can have a wider biodistribution. Thus, importin pathway inhibitor can be used to tune the activity of an anti-DNA antibody such as 3E10 antibody or fragment or variant thereof, by reducing nuclear penetration of non-tumor tissues and further drive equilibrium of antibody or fragment or variant thereof towards tumors.

An exemplary method includes administering to a subject with cancer an effective amount of an importin pathway inhibitor such as ivermectin to reduce nuclear penetration of a nuclear penetrating therapeutic antibody or variant or fragment or fusion protein thereof, without eliminating the ability of the antibody to treat the cancer.

1. Exemplary Nuclear Penetrating Antibodies

a. 3E10

In the early 1990s a murine lupus anti-DNA antibody, 3E10, was tested in experimental vaccine therapy for SLE. These efforts were aimed at developing anti-idiotype antibodies that would specifically bind anti-DNA antibody in SLE patients. However, 3E10 was serendipitously found to penetrate into living cells and nuclei without causing any observed cytotoxicity (Weisbart R H, et al. J Immunol. 1990 144(7): 2653-2658; Zack D J, et al. J Immunol. 1996 157(5): 2082-2088). Studies on 3E10 in SLE vaccine therapy were then supplanted by efforts focused on development of 3E10 as a molecular delivery vehicle for transport of therapeutic molecules into cells and nuclei. 3E10 preferentially binds DNA single-strand tails, inhibits key steps in DNA single-strand and double-strand break repair (Hansen, et al., Science Translational Medicine, 4:157ra142 (2012)). 3E10 can have a V_{H} having an amino acid sequence as shown in SEQ ID NO:6 or 7 and a V_{L} having an amino acid sequence as shown in SEQ ID NO:1 or 2. The 3E10 antibody and its single chain variable fragment which includes a D31N mutation in CDR1 of the V_{H} (3E10 (D31N) scFv) and di- and trivalent fusions thereof penetrate into cells and nuclei and have proven capable of transporting therapeutic protein cargoes attached to the antibody either through chemical conjugation or recombinant fusion. Protein cargoes delivered to cells by 3E10 or 3E10 (D3N) scFv include catalase, p53, and Hsp70 (Weisbart R H, et al. J Immunol. 2000 164: 6020-6026; Hansen J E, et al. Cancer Res. 2007 Feb. 15; 67(4): 1769-74; Hansen J E, et al. Brain Res. 2006 May 9; 1088(1): 187-96). 3E10 (D31N) scFv effectively mediated delivery of Hsp70 to neurons in vivo and this resulted in decreased cerebral infarct volumes and improved neurologic function in a rat stroke model (Zhan X, et al. Stroke. 2010 41(3): 538-43). 3E10 and 3E10 (D31N) scFv and di- and tri-valent fusions thereof, without being conjugated to any therapeutic protein, enhance cancer cell radiosensitivity and chemosensitivity and that this effect is potentiated in cells deficient in DNA repair. Moreover, 3E10 and 3E10 scFv and di- and tri-valent fusions thereof are selectively lethal to cancer cells deficient in DNA repair even in the absence of radiation or chemotherapy. The Food and Drug Administration (FDA) has established a pathway for the development of monoclonal antibodies into human therapies, and 3E10 has already been approved by the FDA for use in a Phase I human clinical trial designed to test the efficacy of 3E10 in experimental vaccine therapy for SLE (Spertini F, et al. J Rheumatol. 1999 26(12): 2602-8).

Experiments indicate that 3E10 (D31N) scFv penetrates cell nuclei by first binding to extracellular DNA or its degradation products and then following them into cell nuclei through the ENT2 nucleoside salvage pathway (Weisbart, Scientific Reports, 5:Article number: 12022 (2015) doi:10.1038/srep12022). When administered to mice and rats 3E10 is preferentially attracted to tissues in which extracellular DNA is enriched, including tumors, regions of ischemic brain in stroke models, and skeletal muscle subject to contractile injury (Weisbart, et al., Sci Rep., 5:12022 (2015), Hansen, et al., J Biol Chem, 282(29):20790-20793 (2007), Weisbart, et al., Mol Immunol, 39(13):783-789 (2003), Zhan, et al., Stroke: A Journal of Cerebral Circulation, 41(3):538-543 (2010)). Thus the presence of extracellular DNA enhances the nuclear uptake of 3E10 (D31N) scFv. Furthermore, 3E10 (D31N) scFv preferentially localizes into tumor cell nuclei in vivo, likely due to increased DNA in the local environment released from ischemic and necrotic regions of tumor.

b. 5C6

5C6 induces γH2AX in BRCA2⁽⁻⁾ but not BRCA2⁽⁺⁾ cells and selectively suppresses the growth of the BRCA2⁽⁻⁾ cells. Mechanistically, 5C6 appears to induce senescence in the BRCA2⁽⁻⁾ cells. Senescence is a well-known response to DNA damage, and DNA damaging agents, including many chemotherapeutics, induce senescence after prolonged exposure (Sliwinska, et al., Mech. Ageing Dev., 130:24-32 (2009); te Poele, et al., Cancer Res. 62:1876-1883 (2002); Achuthan, et al., J. Biol. Chem., 286:37813-37829 (2011)). These observations establish that 5C6 penetrates cell nuclei and damages DNA, and that cells with preexisting defects in DNA repair due to BRCA2 deficiency are more sensitive to this damage than cells with intact DNA repair. See U.S. Published Application No. 2015/0376279. Furthermore, one of skill in the art would appreciate that 5C6 can have a V_{H} having an amino acid sequence as shown in SEQ ID NO:42 and a V_{L} having an amino acid sequence as shown in SEQ ID NO:46.

2. Fragments and Fusion Proteins

In some embodiments, antibody is composed of one or more antigen binding antibody fragments and/or antigen binding fusion proteins of the antibody 3E10 or 5C6, or a variant or humanized form thereof. The antigen binding molecules typically bind to the epitope of 3E10 or 5C6, and can, for example, maintain one or more functions or activities of the full antibody.

Exemplary fragments and fusions include, but are not limited to, single chain antibodies, single chain variable fragments (scFv), di-scFv, tri-scFv, diabody, triabody, teratbody, disulfide-linked Fvs (sdFv), Fab′, F(ab′)₂, Fv, and single domain antibody fragments (sdAb).

In some embodiments, the antibody includes two or more scFv. For example, the antibody can be a scFv or a di-scFv. In some embodiments, each scFv can include one, two, or all three complementarity determining regions (CDRs) of the heavy chain variable region (V_{L}) of 3E10 or 5C6, or a variant thereof. The scFv can include one, two, or all three CDRs of the light chain variable region (V_{L}) of 3E10 or 5C6, or a variant thereof. The molecule can include the heavy chain variable region and/or light chain variable region of 3E10 or 5C6, or a variant thereof.

A single chain variable fragment can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a linker have been developed without significantly disrupting antigen binding or specificity of the binding. The linker is chosen to permit the heavy chain and light chain to bind together in their proper conformational orientation. The linker is usually rich in glycine for flexibility, and typically also includes serine or threonine for solubility. The linker can link, for example, the N-terminus of the V_{H} with the C-terminus of the V_{L}, or vice versa. scFv can also be created directly from subcloned heavy and light chains derived from a hybridoma. In some embodiments, the scFv retains, or improves or increases the specificity of the original immunoglobulin, while removing of the constant regions and introducing the linker.

Exemplary molecules that include two or more single chain variable fragments (scFv) including the light chain variable region (V_{L}) of 3E10 or 5C6, or a variant thereof, and the heavy chain variable region (V_{H}) of 3E10 or 5C6, or a variant thereof of the antibody 3E10 or 5C6 include, but are not limited to, divalent-scFv (di-scFv), trivalent-scFv (tri-scFv), multivalent-scFv (multi-scFv), diabodies, triabodies, tetrabodies, etc., of scFvs.

Divalent single chain variable fragments can be engineered by linking two scFvs. This can be done by producing a single peptide chain with two V_{H} and two V_{L} regions, yielding a di-scFvs referred to as a tandem di-scFv. ScFvs can also be designed with linker peptides that are too short for the two variable regions to fold together (about five amino acids), forcing scFvs to dimerize and form a divalent single chain variable fragment referred to as a diabody. Diabodies have been shown to have dissociation constants up to 40-fold lower than corresponding scFvs, indicating that they have a much higher affinity to their target. Even shorter linkers (one or two amino acids) lead to the formation of trimers (triabodies or tribodies). Tetrabodies have also been produced and have been shown to exhibit an even higher affinity to their targets than diabodies.

The disclosed antibodies include antigen binding antibody fragments and fusion proteins of 3E10 or 5C6 and variants and humanized forms thereof typically bind to the same epitope as the parent antibody 3E10 or 5C6. In some embodiments, the antigen binding molecule is a di-, tri-, or multivalent scFv. Although the antigen binding antibody fragment or fusion protein of the antigen binding molecule can include additional antibody domains (e.g., constant domains, hinge domains, etc.), in some embodiments it does not. For example, 3E10 binds DNA and inhibits DNA repair, which is synthetically lethal to DNA repair-deficient cells. This function is independent of any 3E10 constant regions. By contrast, non-penetrating antibodies such as cetuximab that target extracellular receptors depend in part on Fc-mediated activation of ADCC and complement to exert an effect on tumors. Elimination of the Fc from non-penetrating antibodies could therefore diminish the magnitude of their effect on tumors, but Fc is not required for 3E10 to have an effect on cancer cells. Therefore, 3E10 fragments or fusions that lack an Fc region should be unable to activate ADCC and complement and therefore carry a lower risk of nonspecific side effects.

a. Single Chain Variable Fragments

The single chain variable fragments disclosed herein can include antigen binding fragments of 3E10 or 5C6, or a variant or humanized form thereof. The monoclonal antibody 3E10 and active fragments and exemplary variants thereof that are transported in vivo to the nucleus of mammalian cells without cytotoxic effect are discussed in U.S. Pat. Nos. 4,812,397 and 7,189,396, and U.S. Published Application No. 2014/0050723. Other 3E10 antibody compositions, including fragments and fusions thereof, suitable for use in treating cancer are discussed in, for example, WO 2012/135831, WO 2016/033321, WO 2015/106290, and WO 2016/033324. 5C6 is described in U.S. Published Application No. 2015/0376279.

An scFv includes a light chain variable region (V_{L}) and a heavy chain variable region (V_{H}) joined by a linker. In an example, the linker includes in excess of 12 amino acid residues with (Gly4Ser)3 (SEQ ID NO:50) being one of the more favored linkers for a scFv. In an example, the scFv is a disulfide stabilized Fv (or diFv or dsFv), in which a single cysteine residue is introduced into a FR of VH and a FR of VL and the cysteine residues linked by a disulfide bond to yield a stable Fv. In an example, the scFv is a dimeric scFv (di-scFV), i.e., a protein including two scFv molecules linked by a non-covalent or covalent linkage, e.g., by a leucine zipper domain (e.g., derived from Fos or Jun) or trimeric scFV (tri-scFv). In another example, two scFv's are linked by a peptide linker of sufficient length to permit both scFv's to form and to bind to an antigen, e.g., as described in U.S. Published Application No. 2006/0263367. Additional details are discussed and exemplified below and elsewhere herein.

The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each include four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

The fragments and fusions of antibodies disclosed herein can have bioactivity. For example, the fragments and fusions, whether attached to other sequences or not, can include insertions, deletions, substitutions, or other selected modifications of particular regions or specific amino acids residues. In some embodiments, the activity of the fragment or fusion is not significantly reduced or impaired compared to the nonmodified antibody or antibody fragment.

b. Linkers

The term “linker” as used herein includes, without limitation, peptide linkers. The peptide linker can be any size provided it does not interfere with the binding of the epitope by the variable regions. In some embodiments, the linker includes one or more glycine and/or serine amino acid residues. Monovalent single-chain antibody variable fragments (scFvs) in which the C-terminus of one variable domain are typically tethered to the N-terminus of the other variable domain via a 15 to 25 amino acid peptide or linker. The linker is chosen to permit the heavy chain and light chain to bind together in their proper conformational orientation. Linkers in diabodies, triabodies, etc., typically include a shorter linker than that of a monovalent scFv as discussed above. Di-, tri-, and other multivalent scFvs typically include three or more linkers. The linkers can be the same, or different, in length and/or amino acid composition. Therefore, the number of linkers, composition of the linker(s), and length of the linker(s) can be determined based on the desired valency of the scFv as is known in the art. The linker(s) can allow for or drive formation of a di-, tri-, and other multivalent scFv.

For example, a linker can include 4-8 amino acids. In a particular embodiment, a linker includes the amino acid sequence GQSSRSS (SEQ ID NO:51). In another embodiment, a linker includes 15-20 amino acids, for example, 18 amino acids. In a particular embodiment, the linker includes the amino acid sequence GQSSRSSSGGGSSGGGGS (SEQ ID NO:52). Other flexible linkers include, but are not limited to, the amino acid sequences Gly-Ser, Gly-Ser-Gly-Ser (SEQ ID NO:53), Ala-Ser, Gly-Gly-Gly-Ser (SEQ ID NO:54), (Gly₄-Ser)₂ (SEQ ID NO:55), (Gly₄-Ser)₄ (SEQ ID NO:56), (Gly-Gly-Gly-Gly-Ser)₃ (SEQ ID NO:50), RADAAPGGGGSGGGGSGGGGS (SEQ ID NO:57), and ASTKGPSVFPLAPLESSGS (SEQ ID NO:58).

c. Variants

The antibody or fragment or fusion protein can include an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, or at least 99% identical to the amino acid sequence of the variable heavy chain and/or light chain of 3E10 or 5C6 or a humanized form thereof, and which binds to the epitope of 3E10 or 5C6, is selectively lethal to or selectively increases the radiosensitivity and/or chemosensitivity of cells deficient in DNA repair, or a combination thereof. The antibody or fragment or fusion protein can include a CDR with an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of a CDR of the variable heavy chain and/or light chain of 3E10 or 5C6, and which binds to the epitope of 3E10 or 5C6, is selectively lethal to or selectively increases the radiosensitivity and/or chemosensitivity of cells deficient in DNA repair, or a combination thereof. The determination of percent identity of two amino acid sequences can be determined by BLAST protein comparison. In some embodiments, scFv includes one, two, three, four, five, or all six of the CDRs of the above-described preferred variable domains and which binds to the epitope of 3E10 or 5C6, is selectively lethal to or selectively increases the radiosensitivity and/or chemosensitivity of cells deficient in DNA repair, or a combination thereof.

Predicted complementarity determining regions (CDRs) of the light chain variable sequence for 3E10 or 5C6 are provided below. See also GenBank: AAA65681.1—immunoglobulin light chain, partial [Mus musculus]. Predicted complementarity determining regions (CDRs) of the heavy chain variable sequence for 3E10 and 5C6 are provide above. See, for example, Zack, et al., Immunology and Cell Biology, 72:513-520 (1994) and GenBank Accession number AAA65679.1.

d. 3E10 Antibody Sequences

3E10 can refer to a monoclonal antibody produced by ATCC Accession No. PTA 2439 hybridoma. Mouse and exemplary humanized 3E10 antibody sequences are provided below.

i. 3E10 Light Chain Variable Region

An amino acid sequence for the light chain variable region of 3E10 can be, for example,

(SEQ ID NO: 1)
DIVLTQSPASLAVSLGQRATISCRASKSVSTSSYSYMHWYQQKPGQP
PKLLIKYASYLESGVPARFSGSGSGTDFTLNIHPVEEEDAATYYCQHS
REFPWTFGGGTKLEIK,
or
(SEQ ID NO: 2)
DIVLTQSPASLAVSLGQRATISCRASKSVSTSSYSYMHWYQQKPGQP
PKLLIKYASYLESGVPARFSGSGSGTDFHLNIHPVEEEDAATYYCQHS
REFPWTFGGGTKLELK.

In some embodiments, the complementarity determining regions (CDRs) are defined accordingly to the underlining above (e.g., CDR L1:

(SEQ ID NO: 3)
RASKSVSTSSYSYMH;
CDR L2:
(SEQ ID NO: 4)
YASYLES;
CDR L3:
(SEQ ID NO: 5))
QHSREFPWT.

Other 3E10 light chain sequences are known in the art. See, for example, Zack, et al., J. Immunol., 15; 154(4):1987-94 (1995); GenBank: L16981.1—Mouse Ig rearranged L-chain gene, partial cds; GenBank: AAA65681.1—immunoglobulin light chain, partial [Mus musculus]).

ii. 3E10 Heavy Chain Variable Region

An amino acid sequence for the heavy chain variable region of 3E10 is:

(SEQ ID NO: 6
EVQLVESGGGLVKPGGSRKLSCAASGFTFS [symbol] YGMHWVRQAPEKGLE
WVAYISSGSSTIYYADTVKGRFTISRDNAKNTLFLQMTSLRSEDTAM
YYCARRGLLLDYWGQGTTLTVSS;

Zack, et al., Immunology and Cell Biology, 72:513-520 (1994); GenBank: L16981.1—Mouse Ig rearranged L-chain gene, partial cds; and GenBank: AAA65679.1—immunoglobulin heavy chain, partial [Mus musculus]).

An amino acid sequence for a preferred variant of the heavy chain variable region of 3E10 is:

(SEQ ID NO: 7)
EVQLVESGGGLVKPGGSRKLSCAASGFTFS [symbol] YGMHWVRQAPEKGLE
WVAYISSGSSTIYYADTVKGRFTISRDNAKNTLFLQMTSLRSEDTAM
YYCARRGLLLDYWGQGTTLTVSS.

Amino acid position 31 of the heavy chain variable region of 3E10 has been determined to be influential in the ability of the antibody and fragments thereof to penetrate nuclei and bind to DNA. For example, D31N mutation (bolded and italicized in SEQ ID NOS:6 and 7) in CDR1 penetrates nuclei and binds DNA with much greater efficiency than the original antibody (Zack, et al., Immunology and Cell Biology, 72:513-520 (1994), Weisbart, et al., J. Autoimmun., 11, 539-546 (1998); Weisbart, Int. J. Oncol., 25, 1867-1873 (2004)).

In some embodiments, the complementarity determining regions (CDRs) are defined accordingly to the underlining above (e.g, CDR H1.1 (original sequence): DYGMH (SEQ ID NO:8); CDR H1.2 (with D31N mutation): NYGMH (SEQ ID NO:9); CDR H2: YISSGSSTIYYADTVKG (SEQ ID NO:10); CDR H3: RGLLLDY (SEQ ID NO:11).

iii. Exemplary Mouse scFv

An exemplary mouse 3E10 scFv can have the sequence:

(SEQ ID NO: 12)
AGIHDIVLTQSPASLAVSLGQRATISCRASKSVSTSSYSYMHWYQQK
PGQPPKLLIKYASYLESGVPARFSGSGSGTDFTLNIHPVEEEDAATYY
CQHSREFPWTFGGGTKLEIKRADAAPGGGGSGGGGSGGGGSEVQLV
ESGGGLVKPGGSRKLSCAASGFTFSNYGMHWVRQAPEKGLEWVAYI
SSGSSTIYYADTVKGRFTISRDNAKNTLFLQMTSLRSEDTAMYYCAR
RGLLLDYWGQGTTLTVSSLEQKLISEEDLNSAVDHHHHHH

An exemplary mouse 3E10 di-scFv can have the sequence:

(SEQ ID NO: 13)
AGIHDIVLTQSPASLAVSLGQRATISCRASKSVSTSSYSYMHWYQQK
PGQPPKLLIKYASYLESGVPARFSGSGSGTDFTLNIHPVEEEDAATYY
CQHSREFPWTFGGGTKLEIKRADAAPGGGGSGGGGSGGGGSEVQLV
ESGGGLVKPGGSRKLSCAASGFTFSNYGMHWVRQAPEKGLEWVAYI
SSGSSTIYYADTVKGRFTISRDNAKNTLFLQMTSLRSEDTAMYYCAR
RGLLLDYWGQGTTLTVSSASTKGPSVFPLAPLESSGSDIVLTQSPASL
AVSLGQRATISCRASKSVSTSSYSYMHWYQQKPGQPPKLLIKYASYL
ESGVPARFSGSGSGTDFTLNIHPVEEEDAATYYCQHSREFPWTFGGG
TKLEIKRADAAPGGGGSGGGGSGGGGSEVQLVESGGGLVKPGGSRK
LSCAASGFTFSNYGMHWVRQAPEKGLEWVAYISSGSSTIYYADTVK
GRFTISRDNAKNTLFLQMTSLRSEDTAMYYCARRGLLLDYWGQGTT
LTVSSLEQKLISEEDLNSAVDHHHHHH

iv. Exemplary Humanized 3E10 Variants

-Heavy Chain variable region (variants 2, 6 and
10)
SEQ ID NO: 14
EVQLVESGGGLVQPGGSLRLSCAASGFTFSNYGMHWVRQAPGKGLE
WVSYISSSSSTIYYADSVKGRFTISRDNAKNSLYLQMNSLRAEDTAV
YYCARRGLLLDYWGQGTTVTVSS
-Heavy Chain variable region (variants 3, 7 and
11)
SEQ ID NO: 15
EVQLVESGGGVVQPGGSLRLSCAASGFTFSNYGMHWVRQAPEKGLE
WVSYISSSSSTIYYADSVKGRFTISRDNSKNTLYLQMNSLRAEDTAVY
YCARRGLLLDYWGQGTTVTVSS
-Heavy Chain variable region (variants 4, 8
and 12)
SEQ ID NO: 16
EVQLVESGGGDVKPGGSLRLSCAASGFTFSNYGMHWVRQAPEKGLE
WVSYISSSSSTIYYADSVKGRFTISRDNSKNTLYLQMNSLRAEDTAVY
YCARRGLLLDYWGQGTTVTVSS
-Heavy Chain variable region (variants 13, 16
and 19)
SEQ ID NO: 17
EVQLVESGGGLVQPGGSLRLSCAASGFTFSNYGMHWVRQAPGKGLE
WVSYISSGSSTIYYADSVKGRFTISRDNAKNSLYLQMNSLRAEDTAV
YYCARRGLLLDYWGQGTTVTVSS
-Heavy Chain variable region (variants 14 and 17)
SEQ ID NO: 18
EVQLVESGGGVVQPGGSLRLSCAASGFTFSNYGMHWVRQAPEKGLE
WVSYISSGSSTIYYADSVKGRFTISRDNSKNTLYLQMNSLRAEDTAV
YYCARRGLLLDYWGQGTTVTVSS
-Heavy Chain variable region (variants 15 and 18)
SEQ ID NO: 19
EVQLVESGGGDVKPGGSLRLSCAASGFTFSNYGMHWVRQAPEKGLE
WVSYISSGSSTIYYADSVKGRFTISRDNSKNTLYLQMNSLRAEDTAV
YYCARRGLLLDYWGQGTTVTVSS
-Light Chain variable region (variants 2, 3 and 4)
SEQ ID NO: 20
DIQMTQSPSSLSASLGDRATITCRASKSVSTSSYSYMHWYQQKPGQP
PKLLIKYASYLESGVPSRFSGSGSGTDFTLTISSLQPEDAATYYCQHSR
EFPWTFGGGTKVEIK
-Light Chain variable region (variants 6, 7 and 8)
SEQ ID NO: 21
DIQMTQSPSSLSASLGDRATITCRASKSVSTSSYSYMHWYQQKPGQA
PKLLIKYASYLESGVPSRFSGSGSGTDFTLTISSLQPEDFATYYCQHSR
EFPWTFGQGTKVEIK
-Light Chain variable region (variants 10, 11
and 12)
SEQ ID NO: 22
DIQMTQSPSSLSASVGDRVTITCRASKSVSTSSYSYMHWYQQKPGKA
PKLLIKYASYLESGVPSRFSGSGSGTDFTLTISSLQPEDFATYYCQHSR
EFPWTFGQGTKVEIK
-Light Chain variable region (variants 13, 14
and 15)
SEQ ID NO: 23
DIQMTQSPSSLSASLGDRATITCRASKTVSTSSYSYMHWYQQKPGQP
PKLLIKYASYLESGVPSRFSGSGSGTDFTLTISSLQPEDAATYYCQHSR
EFPWTFGGGTKVEIK
-Light Chain variable region (variants 16, 17
and 18)
SEQ ID NO: 24
DIQMTQSPSSLSASVGDRVTITCRASKTVSTSSYSYMHWYQQKPGKA
PKLLIKYASYLESGVPSRFSGSGSGTDFTLTISSLQPEDFATYYCQHSR
EFPWTFGQGTKVEIK
-Light Chain variable region (variant 19)
SEQ ID NO: 25
DIQMTQSPSSLSASLGDRATITCRASKTVSTSSYSYMHWYQQKPGQA
PKLLIKYASYLESGVPSRFSGSGSGTDFTLTISSLQPEDFATYYCQHSR
EFPWTFGQGTKVEIK
-Variant 2
SEQ ID NO: 26
DIQMTQSPSSLSASLGDRATITCRASKSVSTSSYSYMHWYQQKPGQP
PKLLIKYASYLESGVPSRFSGSGSGTDFTLTISSLQPEDAATYYCQHSR
EFPWTFGGGTKVEIKRADAAPGGGGSGGGGSGGGGSEVQLVESGGG
LVQPGGSLRLSCAASGFTFSNYGMHWVRQAPGKGLEWVSYISSSSST
IYYADSVKGRFTISRDNAKNSLYLQMNSLRAEDTAVYYCARRGLLL
DYWGQGTTVTVSSASTKGPSVFPLAPLESSGSDIQMTQSPSSLSASLG
DRATITCRASKSVSTSSYSYMHWYQQKPGQPPKLLIKYASYLESGVP
SRFSGSGSGTDFTLTISSLQPEDAATYYCQHSREFPWTFGGGTKVEIK
RADAAPGGGGSGGGGSGGGGSEVQLVESGGGLVQPGGSLRLSCAAS
GFTFSNYGMHWVRQAPGKGLEWVSYISSSSSTIYYADSVKGRFTISR
DNAKNSLYLQMNSLRAEDTAVYYCARRGLLLDYWGQGTTVTVSS
-Variant 3
SEQ ID NO: 27
DIQMTQSPSSLSASLGDRATITCRASKSVSTSSYSYMHWYQQKPGQP
PKLLIKYASYLESGVPSRFSGSGSGTDFTLTISSLQPEDAATYYCQHSR
EFPWTFGGGTKVEIKRADAAPGGGGSGGGGSGGGGSEVQLVESGGG
VVQPGGSLRLSCAASGFTFSNYGMHWVRQAPEKGLEWVSYISSSSST
IYYADSVKGRFTISRDNSKNTLYLQMNSLRAEDTAVYYCARRGLLL
DYWGQGTTVTVSSASTKGPSVFPLAPLESSGSDIQMTQSPSSLSASLG
DRATITCRASKSVSTSSYSYMHWYQQKPGQPPKLLIKYASYLESGVP
SRFSGSGSGTDFTLTISSLQPEDAATYYCQHSREFPWTFGGGTKVEIK
RADAAPGGGGSGGGGSGGGGSEVQLVESGGGVVQPGGSLRLSCAAS
GFTFSNYGMHWVRQAPEKGLEWVSYISSSSSTIYYADSVKGRFTISR
DNSKNTLYLQMNSLRAEDTAVYYCARRGLLLDYWGQGTTVTVSS
-Variant 4
SEQ ID NO: 28
DIQMTQSPSSLSASLGDRATITCRASKSVSTSSYSYMHWYQQKPGQP
PKLLIKYASYLESGVPSRFSGSGSGTDFTLTISSLQPEDAATYYCQHSR
EFPWTFGGGTKVEIKRADAAPGGGGSGGGGSGGGGSEVQLVESGGG
DVKPGGSLRLSCAASGFTFSNYGMHWVRQAPEKGLEWVSYISSSSST
IYYADSVKGRFTISRDNSKNTLYLQMNSLRAEDTAVYYCARRGLLL
DYWGQGTTVTVSSASTKGPSVFPLAPLESSGSDIQMTQSPSSLSASLG
DRATITCRASKSVSTSSYSYMHWYQQKPGQPPKLLIKYASYLESGVP
SRFSGSGSGTDFTLTISSLQPEDAATYYCQHSREFPWTFGGGTKVEIK
RADAAPGGGGSGGGGSGGGGSEVQLVESGGGDVKPGGSLRLSCAAS
GFTFSNYGMHWVRQAPEKGLEWVSYISSSSSTIYYADSVKGRFTISR
DNSKNTLYLQMNSLRAEDTAVYYCARRGLLLDYWGQGTTVTVSS
-Variant 6
SEQ ID NO: 29
DIQMTQSPSSLSASLGDRATITCRASKSVSTSSYSYMHWYQQKPGQA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
LVQPGGSLRLSCAASGFTFSNYGMHWVRQAPGKGLEWVSYISSSSST
IYYADSVKGRFTISRDNAKNSLYLQMNSLRAEDTAVYYCARRGLLL
DYWGQGTTVTVSSASTKGPSVFPLAPLESSGSDIQMTQSPSSLSASLG
DRATITCRASKSVSTSSYSYMHWYQQKPGQAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGLVQPGGSLRLSCAAS
GFTFSNYGMHWVRQAPGKGLEWVSYISSSSSTIYYADSVKGRFTISR
DNAKNSLYLQMNSLRAEDTAVYYCARRGLLLDYWGQGTTVTVSS
-Variant 7
SEQ ID NO: 30
DIQMTQSPSSLSASLGDRATITCRASKSVSTSSYSYMHWYQQKPGQA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
VVQPGGSLRLSCAASGFTFSNYGMHWVRQAPEKGLEWVSYISSSSST
IYYADSVKGRFTISRDNSKNTLYLQMNSLRAEDTAVYYCARRGLLL
DYWGQGTTVTVSSASTKGPSVFPLAPLESSGSDIQMTQSPSSLSASLG
DRATITCRASKSVSTSSYSYMHWYQQKPGQAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGVVQPGGSLRLSCAAS
GFTFSNYGMHWVRQAPEKGLEWVSYISSSSSTIYYADSVKGRFTISR
DNSKNTLYLQMNSLRAEDTAVYYCARRGLLLDYWGQGTTVTVSS
-Variant 8
SEQ ID NO: 31
DIQMTQSPSSLSASLGDRATITCRASKSVSTSSYSYMHWYQQKPGQA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
DVKPGGSLRLSCAASGFTFSNYGMHWVRQAPEKGLEWVSYISSSSST
IYYADSVKGRFTISRDNSKNTLYLQMNSLRAEDTAVYYCARRGLLL
DYWGQGTTVTVSSASTKGPSVFPLAPLESSGSDIQMTQSPSSLSASLG
DRATITCRASKSVSTSSYSYMHWYQQKPGQAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGDVKPGGSLRLSCAAS
GFTFSNYGMHWVRQAPEKGLEWVSYISSSSSTIYYADSVKGRFTISR
DNSKNTLYLQMNSLRAEDTAVYYCARRGLLLDYWGQGTTVTVSS
-Variant 10
SEQ ID NO: 32
DIQMTQSPSSLSASVGDRVTITCRASKSVSTSSYSYMHWYQQKPGKA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
LVQPGGSLRLSCAASGFTFSNYGMHWVRQAPGKGLEWVSYISSSSST
IYYADSVKGRFTISRDNAKNSLYLQMNSLRAEDTAVYYCARRGLLL
DYWGQGTTVTVSSASTKGPSVFPLAPLESSGSDIQMTQSPSSLSASVG
DRVTITCRASKSVSTSSYSYMHWYQQKPGKAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGLVQPGGSLRLSCAAS
GFTFSNYGMHWVRQAPGKGLEWVSYISSSSSTIYYADSVKGRFTISR
DNAKNSLYLQMNSLRAEDTAVYYCARRGLLLDYWGQGTTVTVSS
-Variant 11
SEQ ID NO: 33
DIQMTQSPSSLSASVGDRVTITCRASKSVSTSSYSYMHWYQQKPGKA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
VVQPGGSLRLSCAASGFTFSNYGMHWVRQAPEKGLEWVSYISSSSST
IYYADSVKGRFTISRDNSKNTLYLQMNSLRAEDTAVYYCARRGLLL
DYWGQGTTVTVSSASTKGPSVFPLAPLESSGSDIQMTQSPSSLSASVG
DRVTITCRASKSVSTSSYSYMHWYQQKPGKAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGVVQPGGSLRLSCAAS
GFTFSNYGMHWVRQAPEKGLEWVSYISSSSSTIYYADSVKGRFTISR
DNSKNTLYLQMNSLRAEDTAVYYCARRGLLLDYWGQGTTVTVSS
-Variant 12
SEQ ID NO: 34
DIQMTQSPSSLSASVGDRVTITCRASKSVSTSSYSYMHWYQQKPGKA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
DVKPGGSLRLSCAASGFTFSNYGMHWVRQAPEKGLEWVSYISSSSST
IYYADSVKGRFTISRDNSKNTLYLQMNSLRAEDTAVYYCARRGLLL
DYWGQGTTVTVSSASTKGPSVFPLAPLESSGSDIQMTQSPSSLSASVG
DRVTITCRASKSVSTSSYSYMHWYQQKPGKAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGDVKPGGSLRLSCAAS
GFTFSNYGMHWVRQAPEKGLEWVSYISSSSSTIYYADSVKGRFTISR
DNSKNTLYLQMNSLRAEDTAVYYCARRGLLLDYWGQGTTVTVSS
-Variant 13
SEQ ID NO: 35
DIQMTQSPSSLSASLGDRATITCRASKTVSTSSYSYMHWYQQKPGQP
PKLLIKYASYLESGVPSRFSGSGSGTDFTLTISSLQPEDAATYYCQHSR
EFPWTFGGGTKVEIKRADAAPGGGGSGGGGSGGGGSEVQLVESGGG
LVQPGGSLRLSCAASGFTFSNYGMHWVRQAPGKGLEWVSYISSGSST
IYYADSVKGRFTISRDNAKNSLYLQMNSLRAEDTAVYYCARRGLLL
DYWGQGTTVTVSSASTKGPSVFPLAPLESSGSDIQMTQSPSSLSASLG
DRATITCRASKTVSTSSYSYMHWYQQKPGQPPKLLIKYASYLESGVP
SRFSGSGSGTDFTLTISSLQPEDAATYYCQHSREFPWTFGGGTKVEIK
RADAAPGGGGSGGGGSGGGGSEVQLVESGGGLVQPGGSLRLSCAAS
GFTFSNYGMHWVRQAPGKGLEWVSYISSGSSTIYYADSVKGRFTISR
DNAKNSLYLQMNSLRAEDTAVYYCARRGLLLDYWGQGTTVTVSS
-Variant 14
SEQ ID NO: 36
DIQMTQSPSSLSASLGDRATITCRASKTVSTSSYSYMHWYQQKPGQP
PKLLIKYASYLESGVPSRFSGSGSGTDFTLTISSLQPEDAATYYCQHSR
EFPWTFGGGTKVEIKRADAAPGGGGSGGGGSGGGGSEVQLVESGGG
VVQPGGSLRLSCAASGFTFSNYGMHWVRQAPEKGLEWVSYISSGSS
TIYYADSVKGRFTISRDNSKNTLYLQMNSLRAEDTAVYYCARRGLLL
DYWGQGTTVTVSSASTKGPSVFPLAPLESSGSDIQMTQSPSSLSASLG
DRATITCRASKTVSTSSYSYMHWYQQKPGQPPKLLIKYASYLESGVP
SRFSGSGSGTDFTLTISSLQPEDAATYYCQHSREFPWTFGGGTKVEIK
RADAAPGGGGSGGGGSGGGGSEVQLVESGGGVVQPGGSLRLSCAAS
GFTFSNYGMHWVRQAPEKGLEWVSYISSGSSTIYYADSVKGRFTISR
DNSKNTLYLQMNSLRAEDTAVYYCARRGLLLDYWGQGTTVTVSS
-Variant 15
SEQ ID NO: 37
DIQMTQSPSSLSASLGDRATITCRASKTVSTSSYSYMHWYQQKPGQP
PKLLIKYASYLESGVPSRFSGSGSGTDFTLTISSLQPEDAATYYCQHSR
EFPWTFGGGTKVEIKRADAAPGGGGSGGGGSGGGGSEVQLVESGGG
DVKPGGSLRLSCAASGFTFSNYGMHWVRQAPEKGLEWVSYISSGSS
TIYYADSVKGRFTISRDNSKNTLYLQMNSLRAEDTAVYYCARRGLLL
DYWGQGTTVTVSSASTKGPSVFPLAPLESSGSDIQMTQSPSSLSASLG
DRATITCRASKTVSTSSYSYMHWYQQKPGQPPKLLIKYASYLESGVP
SRFSGSGSGTDFTLTISSLQPEDAATYYCQHSREFPWTFGGGTKVEIK
RADAAPGGGGSGGGGSGGGGSEVQLVESGGGDVKPGGSLRLSCAAS
GFTFSNYGMHWVRQAPEKGLEWVSYISSGSSTIYYADSVKGRFTISR
DNSKNTLYLQMNSLRAEDTAVYYCARRGLLLDYWGQGTTVTVSS
-Variant 16
SEQ ID NO: 38
DIQMTQSPSSLSASVGDRVTITCRASKTVSTSSYSYMHWYQQKPGKA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
LVQPGGSLRLSCAASGFTFSNYGMHWVRQAPGKGLEWVSYISSGSS
TIYYADSVKGRFTISRDNAKNSLYLQMNSLRAEDTAVYYCARRGLL
LDYWGQGTTVTVSSASTKGPSVFPLAPLESSGSDIQMTQSPSSLSASV
GDRVTITCRASKTVSTSSYSYMHWYQQKPGKAPKLLIKYASYLESGV
PSRFSGSGSGTDFTLTISSLQPEDFATYYCQHSREFPWTFGQGTKVEIK
RADAAPGGGGSGGGGSGGGGSEVQLVESGGGLVQPGGSLRLSCAAS
GFTFSNYGMHWVRQAPGKGLEWVSYISSGSSTIYYADSVKGRFTISR
DNAKNSLYLQMNSLRAEDTAVYYCARRGLLLDYWGQGTTVTVSS
-Variant 17
SEQ ID NO: 39
DIQMTQSPSSLSASVGDRVTITCRASKTVSTSSYSYMHWYQQKPGKA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
VVQPGGSLRLSCAASGFTFSNYGMHWVRQAPEKGLEWVSYISSGSS
TIYYADSVKGRFTISRDNSKNTLYLQMNSLRAEDTAVYYCARRGLLL
DYWGQGTTVTVSSASTKGPSVFPLAPLESSGSDIQMTQSPSSLSASVG
DRVTITCRASKTVSTSSYSYMHWYQQKPGKAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGVVQPGGSLRLSCAAS
GFTFSNYGMHWVRQAPEKGLEWVSYISSGSSTIYYADSVKGRFTISR
DNSKNTLYLQMNSLRAEDTAVYYCARRGLLLDYWGQGTTVTVSS
-Variant 18
SEQ ID NO: 40
DIQMTQSPSSLSASVGDRVTITCRASKTVSTSSYSYMHWYQQKPGKA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
DVKPGGSLRLSCAASGFTFSNYGMHWVRQAPEKGLEWVSYISSGSS
TIYYADSVKGRFTISRDNSKNTLYLQMNSLRAEDTAVYYCARRGLLL
DYWGQGTTVTVSSASTKGPSVFPLAPLESSGSDIQMTQSPSSLSASVG
DRVTITCRASKTVSTSSYSYMHWYQQKPGKAPKLLIKYASYLESGVP
SRFSGSGSGTDFTLTISSLQPEDFATYYCQHSREFPWTFGQGTKVEIK
RADAAPGGGGSGGGGSGGGGSEVQLVESGGGDVKPGGSLRLSCAAS
GFTFSNYGMHWVRQAPEKGLEWVSYISSGSSTIYYADSVKGRFTISR
DNSKNTLYLQMNSLRAEDTAVYYCARRGLLLDYWGQGTTVTVSS
-Variant 19
SEQ ID NO: 41
DIQMTQSPSSLSASLGDRATITCRASKTVSTSSYSYMHWYQQKPGQA
PKLLIKYASYLESGVPSRFSGSGSGTDFTLTISSLQPEDFATYYCQHSR
EFPWTFGQGTKVEIKRADAAPGGGGSGGGGSGGGGSEVQLVESGGG
LVQPGGSLRLSCAASGFTFSNYGMHWVRQAPGKGLEWVSYISSGSS
TIYYADSVKGRFTISRDNAKNSLYLQMNSLRAEDTAVYYCARRGLL
LDYWGQGTTVTVSSASTKGPSVFPLAPLESSGSDIQMTQSPSSLSASL
GDRATITCRASKTVSTSSYSYMHWYQQKPGQAPKLLIKYASYLESGV
PSRFSGSGSGTDFTLTISSLQPEDFATYYCQHSREFPWTFGQGTKVEIK
RADAAPGGGGSGGGGSGGGGSEVQLVESGGGLVQPGGSLRLSCAAS
GFTFSNYGMHWVRQAPGKGLEWVSYISSGSSTIYYADSVKGRFTISR
DNAKNSLYLQMNSLRAEDTAVYYCARRGLLLDYWGQGTTVTVSS

e. 5C6 Antibody Sequences

5C6 refers to a monoclonal anti-DNA antibody with nucleolytic activity produced by a hybridoma from MRL/lpr lupus mouse model as described in Noble et al., 2014, Sci Rep 4:5958 doi: 10.1038/srep05958. Mouse 5C6 sequences are provided below.

i. 5C6 Light Chain Variable Region

An amino acid sequence for the kappa light chain variable region (VL) of mAb 5C6 is:

(SEQ ID NO: 42)
DIVLTQSPASLAAVSLGERATISYRASKSVSTSGYSYMHWNQQKPGQ
APRLLIYLVSNLESGVPARFSGSGSGTDFTLNIHPVEEEDAATYYCQH
IRELDTFFGGGTKLEIK.

In some embodiments, the complementarity determining regions (CDRs) are defined accordingly to the underlining above (e.g., CDR L1:

(SEQ ID NO: 43)
RASKSVSTSGYSYMH;
CDR L2:
(SEQ ID NO: 44)
LVSNLES;
CDR L3:
(SEQ ID NO: 45))
QHIRELDTF.

ii. 5C6 Heavy Chain Variable Region

An amino acid sequence for the heavy chain variable region (VH) of mAb 5C6 is:

(SEQ ID NO: 46)
QLKLVESGGGLVKPGGSLKLSCAASGFTFSSYTMSWVRQTPAKRLE
WVATISSGGGSTYYPDSVKGRFTISRDNARNTLYLQMSSLRSEDTAM
YYCARRAYSKRGAMDYWGQGTSVTVSS.

In some embodiments, the complementarity determining regions (CDRs) are defined accordingly to the underlining above (e.g., CDR H1: SYTMS (SEQ ID NO:47); CDR H2: TISSGGGSTYYPDSVKG (SEQ ID NO:48); CDR H3: RAYSKRGAMDY (SEQ ID NO:49)).

### EXAMPLES

### Example 1: Nuclear Uptake of Variant 13 is Independent of Endocytosis

Materials and Methods

Reagents

Unless otherwise stated, all cell culture reagents were obtained from Invitrogen (Carlsbad, Calif., USA). Dynasore, Methyl beta-cyclodextrin, EIPA, Chlorpromazine, Ivermectin, Importazole, leptomycin B, Filipin, and Mifeprestone were purchased from Sigma Aldrich. Concentrated stock solutions of all compound were prepared either in DMSO or PBS. For all pre-incubation experiments, the target final concentration of drug was prepared in the respective cell culture medium, and sterile-filtered immediately prior to experimentation. Protein L, protein L primary antibody and goat, anti-chicken secondary antibodies were acquired from Invitrogen.

Cells

DLD-1 colon cancer cells (Horizon Discovery Ltd) were cultivated in RPMI1640 containing 10% v/v FBS, and A549 lung carcinoma cells (ATCC) and MCF7 breast cancer cells (ATCC) were maintained in high-glucose DMEM supplemented with 10% v/v FBS. All cell lines were maintained in a pre-humidified atmosphere containing 5% v/v CO2 and used within ten passages.

Variant 13 Protein Expression and Purification

3E10 antibody sequences are provided above. Expression and purification of variant 13 (e.g., SEQ ID NOS:17, 23, and 35) from a CHO cell system was performed using a previously-described approach (Rattray et al., Biochem Biophys Res Commun 496, 858-864 (2018)). Purity and stability of variant 13 was verified using a combination of SDS-PAGE, and size-exclusion chromatography (SEC-HPLC).

Protein L Immunodetection of Antibody Cell Penetration

For all experiments, cells were cultivated overnight to allow for attachment, followed by pretreatment with either control medium (absence of inhibitor), or medium containing inhibitor for durations previously-reported in the literature (see Table 5, below).

TABLE 5
Inhibitors

| | | | | EFFECTOR/ | |
|---|---|---|---|---|---|
| | | CONCENTRATION | DURATION | TRAFFICKING | |
| COMPOUND | ABBREV. | (μM) | (HOURS) | MECHANISM | REF. |
| CHLORPROMAZINE | CPZ | 10-100 | 1 | AP2, CME | Dutta, et al., Cell. Log., |
| | | | | | 2: 203-208 (2012) |
| DYNASORE | Dyn | 10-100 | 1 | CME, dynamin | Macia et al., Dev. Cell, |
| | | | | GTPase inhibitor | 10: 839-850 (2006) |
| METHYL-B- | MβCD | 5,000-10,000 | 1 | Cholesterol | Dutta, et al., Cell. Log., |
| CYCLODEXTRIN | | | | sequestration, CIE | 2: 203-208 (2012) |
| 5-(N-ETHYL-N- | EIPA | 10-100 | 1 | Na⁺/H⁺ exchange, | Gekle et al., J of |
| ISOPROPYL)- | | | | Macropinocytosis | Physio., 520: 709-721 |
| AMILORIDE | | | | | (1999). |
| SODIUM AZIDE | NaN₃ | 10,000-100,000 | 0.5-1 | Energy-dependent | Cooper, et al., J. Biol. |
| | | | | | Chem., 281: 16563- |
| | | | | | 16569 (2006) |
| TEMPERATURE | T | N/A | 0.5-1 | Energy-dependent | |
| LEPTOMYCIN B | LMB | 0.001-0.02 | 1-4 | CRM1, | van der Watt et al., Mol. |
| | | | | Nuclear export | Can. Ther., 15: 560-573 |
| | | | | | (2016), Lundberg et al., |
| | | | | | Antiviral Res., 100: 662- |
| | | | | | 672 (2013) |
| IVERMECTIN | — | 10-100 | 1-4 | Importin α/β, | (Wagstaff et al., Biochem. |
| | | | | Nuclear import | J., 443, 851-856 (2012), |
| | | | | | van der Watt et al., Mol. |
| | | | | | Can. Ther., 15: 560-573 |
| | | | | | (2016) |
| MIFEPRISTONE | — | 10-200 | 1-4 | Importin α/β-NLS | Lundberg et al., |
| | | | | interaction, | Antiviral Res., 100: 662- |
| | | | | Nuclear import | 672 (2013), Wagstaff et |
| | | | | | al., J. of Biom. Screen., |
| | | | | | 16, 192-200 (2011) |
| IMPORTAZOLE | — | 10-100 | 1-4 | Nuclear import | Soderholm et al., ACS |
| | | | | | Chem. Bio., 6, 700-708 |
| | | | | | (2011) |

For endocytosis inhibition experiments, cells were cultivated in serum-free medium containing 1% v/v BSA to deplete endogenous serum levels. Following preincubation with inhibitors, cells were treated with either control medium or 10 μM variant 13, and the same dose of small molecule inhibitor for a further 30 minutes. For all experiments, cells were washed thrice, followed by immediate fixation with pre-chilled ice-cold ethanol. Subsequently, treated cells were probed using a protein L immunodetection approach to probe variant 13 nuclear uptake (Rattray et al., Biochem Biophys Res Commun 496, 858-864 (2018)). All monolayers were immediately imaged following color development on an Evos Fl microscope.

Results

Previous reports have considered the role of energy-dependent, endocytic trafficking in cellular uptake of ANAs. A panel of small molecule inhibitors of clathrin-dependent and -independent endocytosis were used to test the role of endocytosis in variant 13 cellular and nuclear penetration. Doses of inhibitors used in this study were selected to ensure minimal toxicity to the cells. Cells were pre-treated with inhibitors prior to the addition of variant 13 and evaluation of its cellular penetration by protein L immunostain. The results indicate that inhibition of endocytosis did not impact variant 13 cellular and nuclear penetration.

### Example 2: The Sequence of 3E10 VL has a Putative Bipartite Classic Nuclear Localization Signal

Materials and Methods

In Silico Prediction of Nuclear Localization Sequences

The “cNLS Mapper” program, an opensource in silico NLS prediction software, was used to screen for any sequences that may be similar to classic nuclear localization signals (NLS). A cut-off score of 4.0 was selected (higher values correlate with increased likelihood of representing an NLS). This program is based on Kosugi et al., (2009) PNAS, 106, 10171-10176.

Amino acid sequences of antibody VH and VL were input into online cNLS Mapper. NLS Mapper does not rely on comparison with cNLS sequence libraries, but uses contribution scores from amino acid to predict potential cNLS presence within an amino acid sequence (Kosugi et al., Proc Natl Acad Sci USA 106, 10171-10176 (2009)). G1-5 sequence is available at Genbank AF289183.1. H241, G2-6, 2C10, and G5-8 sequences are presented in reference (Im et al., Mol Immunol 67, 377-387 (2015)). 3D8 sequence is available in patent WO 2010/056043.

3E10 Sequences

3E10 sequences are provided above. The analysis below is focused on humanized variant 10 (e.g., SEQ ID NOS:14, 22, and 32) and variant 13 (e.g., SEQ ID NOS:17, 23, and 35).

Results

A range of humanized 3E10 variants were generated with subtle differences in framework and CDR sequences. In evaluating the ability of these variants to carry out the original functions of 3E10 (D31N) di-scFv, a select number of the variants were found to penetrate cell nuclei more efficiently than the original 3E10 (D31N) di-scFv, while others were found to have lost the ability to penetrate nuclei. In particular, variants 10 and 13 penetrated nuclei very well compared to the prototype.

The sequences of the VL and VH of 3E10 and the new variants were compared to determine if key changes could be identified that are responsible for an increase in efficiency of nuclear penetration. The NLS Mapper software, which screens and scores amino acid sequences for potential classic NLS sequences (Kosugi et al., Proc Nat Acad Sci USA 106, 10171-10176 (2009)), was used to evaluate the 3E10 VH and VL. The entire sequences were analyzed, with a cut-off score of 4.0 (higher scores equate to greater probability of a cNLS). Under these parameters, no cNLS was predicted in the 3E10 VH sequence. However, a potential bipartite cNLS with score 4.6 was found in the 3E10 VL sequence, spanning the CDR1, framework 2, and first amino acid of CDR2 (Table 6)

3E10 VL possible bipartite NLS:
(SEQ ID NO: 59)
RASKSVSTSSYSYMHWYQQKPGQPPKLLIKY

When the sequence of variant 13 was screened, the CDR1 and framework 2 sequences were again identified as a possible NLS, this time with a score of 4.8, with the S-T change present in CDR1 of variant 13 increasing the likelihood of this sequence being an NLS.

Variant 13 possible bipartite NLS:
(SEQ ID NO: 60)
RASKTVSTSSYSYMHWYQQKPGQPPKLLIKY

When the sequence of variant 10 was screened, the same apparent NLS was again identified. Additionally, an extended sequence involving part of framework 1, CDR1, and part of framework 2 was identified as a stronger possible NLS, this time with a score of 5.9.

Variant 10 possible bipartite NLS:
(SEQ ID NO: 61)
RVTITCRASKSVSTSSYSYMHWYQQKPGKAPKL

The sequence of 3E10 VL has a putative bipartite classic nuclear localization signal that is relatively conserved across a panel of known nuclear-penetrating anti-DNA autoantibodies. Identification of a possible bipartite NLS in 3E10 has important implications in understanding the mechanism by which 3E10 localizes to the nucleus. Presence of an NLS indicates that 3E10 may cross the nuclear envelope via the nuclear import pathway (importin pathway), and as discussed further below, follow-up studies using an inhibitor of the importin pathway support this conclusion.

An NIH Basic Local Alignment Search Tool search on this putative cNLS yielded many anti-DNA antibody matches, and similar sequences were identified in the VL of several other known nuclear-localizing anti-dsDNA autoantibodies. Specifically, cNLS Mapper identified nearly identical putative cNLS sequences in VL of the nuclear-localizing G1-5, G2-6, and H241 anti-dsDNA autoantibodies. In contrast, this sequence is absent in anti-dsDNA autoantibodies that are known to depend on VH for nuclear localization (2C10), that localize in the cytoplasm (3D8), or that cannot penetrate cells (G5-8) (Im et al., Mol Immunol 67, 377-387 (2015), Lee et al., Biorg Med Chem 15, 2016-23 (2007); Kim et al., J Biol Chem 281, 15287-95 (2006); Yang et al., Cell Mol Life Sci 66, 1985-97 (2009)) (Table 6). Taken together, these findings are supportive of a potential cNLS motif that is conserved within the sequence of some nuclear-penetrating anti-dsDNA autoantibodies.

TABLE 6
VL Sequences of cell-penetrating anti-DNA autoantibodies.
CDR1 + framework 2 sequence are in bold. Additional important sequence
for variant 10 is in bold/italics. The VL and VH amino acid sequences of
anti-dsDNA autoantibodies that localize to nuclei (3E10, G1-5, H241, G2-6,
and 2C10), localize to cytoplasm (3D8), or cannot penetrate cells (G5-8)
were screened for the presence of a cNLS by cNLS Mapper, with cutoff
score 4.0. A nearly identical potential bipartite cNLS was identified in VL of
all of the nuclear-localizing antibodies except 2C10, which is known to use
VH for cellular penetration (Im et al., Mol Immunol 67, 377-387 (2015)).

| | VL/Potential VL cNLS Sequence | VL | Nuclear |
|---|---|---|---|
| Name/Source | (bold with underlining) | cNLS? | localizing? |
| 3E10 | DIVLTQSPASLAVSLGQRATISCRASKSVSTSSYS | Yes | Yes |
| MRL/lpr | YMHWYQQKPGQPPKLLIKYASYLESGVPARFS | | |
| | GSGSGTDFTLNIHPVEEEDAATYYCQHSREFPWT | | |
| | FGGGTKLEIK (SEQ ID NO: 1) | | |
| Var 13 | DIQMTQSPSSLSASLGDRATITCRASKTVSTSSYS | Yes | Yes |
| Humanized | YMHWYQQKPGQPPKLLIKYASYLESGVPSRFS | | |
| | GSGSGTDFTLTISSLQPEDAATYYCQHSREFPWTF | | |
| | GGGTKVEIK (SEQ ID NO: 23) | | |
| Var 10 | DIQMTQSPSSLSASVGD [symbol] RASKSVSTSSYS | Yes | Yes |
| Humanized | YMHWYQQKPGKAPKLLIKYASYLESGVPSRFS | | |
| | GSGSGTDFTLTISSLQPEDFATYYCQHSREFPWTF | | |
| | GQGTKVEIK (SEQ ID NO: 22) | | |
| 5C6 | DIVLTQSPASLAAVSLGERATISYRASKSVSTSGY | Yes | Yes |
| MRL/lpr | SYMHWNQQKPGQAPRLLIYLVSNLESGVPARF | | |
| | SGSGSGTDFTLNIHPVEEEDAATYYCQHIRELDTF | | |
| | FGGTKLEIK (SEQ ID NO: 42) | | |
| G1-5 | DVVMTQSPASLAVSLGQRATISCRASKSVSTSSY | Yes | Yes |
| MRL/lpr | NYMHWHQQKPGQPPKLLIKYASYLESGVPARF | | |
| GenBank | SGSGSGTDFTLNIHPVEEEDAATYYCHHSREFPW | | |
| AF289183.1 | TFGGGTKLEIKRA (SEQ ID NO: 63) | | |
| H241 | RASKSVSTSNYSYMYWYQQKPGQPPKLLIKY | Yes | Yes |
| | (SEQ ID NO: 68) | | |
| G2-6 | RASKSVSTSSYNYIHWHQQKPGQPPKLLIKY | Yes | Yes |
| | (SEQ ID NO: 69) | | |
| 2C10 | — | No (uses | Yes |
| | | VH) | |
| 3D8 | DLVMSQSPSSLAVSAGEKVTMSCKSSQSLFNSRT | No | No, |
| MRL/lpr | RKNYLAWYQQKPGQSPKLLIYWASTRESGVPD | | localizes to |
| Genbank | RFTGSGSGTDFTLTISSVQAEDLAVYYCKQSYYH | | cytoplasm |
| K1939261.1 | MYTFGSGTKLEIK (SEQ ID NO: 64) | | |
| 4H2 | DIVLTQSPATLSVTPGDRVSLSCRASQSISNYLH | No | No, |
| MRL/lpr | WYQQKSHESPRLLIKYASQSISGIPSRFSGSGSGT | | localizes to |
| | DFTLSIISVETEDFGMYFCQQSNSWPLTFGAGTKL | | cytoplasm |
| | ELK (SEQ ID NO: 62) | | |
| G5-8 | — | No | No, cannot |
| | | | penetrate |
| | | | cells |

### Example 3: Ivermectin Inhibits Nuclear Penetration by Variant 13 and Human SLE Antibodies

Materials and Methods

DLD-1 and A549 cells were treated with 10 μM variant 13 co-incubated with 0, 10, 25, or 50 μM concentrations of ivermectin. Relative intensity of protein L nuclear staining as compared to amount of stain in cells treated without ivermectin was also quantified by ImageJ.

Live MCF7 breast cancer cells were treated with control or rhodamine-labeled variant 13 (Rh-variant 13)+/−50 μM ivermectin and were then visualized under light and fluorescence microscopy. Ivermectin inhibited cellular/nuclear uptake of Rh-variant 13.

Results

3E10 scFv was previously shown to utilize the ENT2 nucleoside salvage pathway to penetrate cells (Hansen et al., J Biol Chem 282, 20790-20793 (2007); Wang et al., Biochem Pharmacol 86,10.1016/j.bcp.2013.1008.1063 (2013); Boswell-Casteel and Hays, Nucleosides Nucleotide Nucleic Acids 36, 7-30 (2017)). Nuclear localizing signal (NLS)-like-motifs may facilitate nuclear penetration of some antinuclear antibodies (ANA) (Im et al., Mol Immunol 67, 377-387 (2015); Im et al., Animal Cells and Systems 21, 382-387 (2017); Deng et al., Int Immunol 12, 415-423 (2000)). NLS-based nuclear import most commonly involves the importin α/β pathway, but the role of this pathway in nuclear uptake of ANAs does not appear to have been experimentally tested previously.

The identification of a bipartite NLS in the 3E10 VL raised the possibility that the importin pathway is involved in the mechanism of nuclear localization by 3E10. Small molecule drugs including ivermectin, mifepristone, and importazole, were used to evaluate the role of the importin α/β pathway in the mechanism of variant 13 nuclear import. Ivermectin and mifepristone both inhibit the importin α/β pathway, and importazole has been reported to selectively inhibit the activity of importin β without perturbing transportin function (Wagstaff et al., Biochem J 443, 851-856 (2012); Wagstaff et al., J Biomol Screen 16, 192-200 (2011); van der Watt et al., Mol Cancer Ther 15, 560-573 (2016); Soderholm et al., ACS Chem Biol 6, 700-708 (2011); Lundberg et al., Antiviral Res 100, 662-672 (2013)). The impact of each inhibitor on nuclear uptake of variant 13 in DLD1 colon and A549 lung cancer cells was tested.

In this Example, the effect of ivermectin; (Wagstaff et al., Biochemical Journal 2012 443(3): 851-856)) on the ability of variant 13 to penetrate into the nuclei of cells was evaluated. DLD1 colon cancer cells and A549 lung cancer cells were treated with variant 13 in the presence of 0-50 μM ivermectin. Cells were subsequently washed, fixed, and immunostained using protein L for detection of variant 13. Representative micrographs of the cells after staining demonstrated a dose-dependent reduction in nuclear penetration by variant 13 caused by ivermectin. Relative intensity of nuclear staining as compared to amount of stain in cells treated without ivermectin was also quantified by ImageJ (FIG. 1). Ivermectin also inhibited cellular/nuclear uptake of a rhodamine-labeled variant 13 into MCF7 breast cancer cells.

### Example 4: Importazole Inhibits Nuclear Penetration by Variant 13

Materials and Methods

DLD-1 cells and A549 cells were treated with 10 μM variant 13 co-incubated with 0, 10, 25, or 50 μM concentrations of importazole and then immunostained for protein L.

Live DLD1 cancer cells were treated with control or rhodamine-labeled variant 13 (Rh-variant 13)+/−50 μM importazole and were then visualized under light and fluorescence microscopy.

Results

The identification of a bipartite NLS in the 3E10 VL raised the possibility that the importin pathway is involved in the mechanism of nuclear localization by 3E10. To test this, the effect of a nuclear import inhibitor that has been reported to have specificity for this pathway (importazole; Soderholm et al., ACS Chem Biol 20116(7): 700-8) on the ability of variant 13 to penetrate into the nuclei of DLD1 cells and A549 cells was evaluated. DLD1 and A549 cells were treated with variant 13 in the presence of 0-50 μM importazole. Cells were subsequently washed, fixed, and immunostained using protein L for detection of variant 13. Representative micrographs of the cells after staining demonstrated a reduction in nuclear penetration by variant 13 caused by importazole. Importazole also inhibited nuclear uptake of a rhodamine-labeled variant 13 into DLD1 cells.

### Example 5: Mifepristone Inhibits Nuclear Penetration by Variant 13

Materials and Methods

DLD-1 cells and A549 cells were treated with 10 μM variant 13 co-incubated with 0, 10, 25, or 50 μM concentrations of mifepristone and then immunostained for protein L.

Results

The identification of a bipartite NLS in the 3E10 VL raised the possibility that the importin pathway is involved in the mechanism of nuclear localization by 3E10. To test this, the effect of a nuclear import inhibitor that has been reported to have specificity for this pathway (mifepristone; Wagstaff et al., J Biomol Screen 2011 16(2): 192-200) on the ability of variant 13 (Variant 13) to penetrate into the nuclei of DLD1 cells and A549 cells was evaluated. DLD1 and A549 cells were treated with variant 13 in the presence of 0-50 μM mifepristone. Cells were subsequently washed, fixed, and immunostained using protein L for detection of variant 13. Representative micrographs of the cells after staining demonstrated a reduction in nuclear penetration by variant 13 caused by mifepristone.

### Example 6: Knockdown of Importin β1 Reduce 3E10 Nuclear Localization

Materials and Methods

Generation and Verification of Importin-β1 knockdowns

Importin-β1 (KPNB1) knockdown in DLD1 cells were generated using control (D-001206-14-05) and KPNB1 (importin β1, M-017523-01-0005) siRNA (Dharmacon). DLD1 cells were seeded into 24-well (western blot) or 96-well plates (cell penetration assays) overnight. Cell monolayers were transfected with siRNA, followed by media replacement at 24 hours, and evaluation at multiple time points post-transfection. Importin β1 expression was then evaluated by western blotting of cell lysates.

Results

Knockdown of importin 1 in DLD1 cells by siRNA was next performed to further probe the role of this pathway in modulating variant 13 nuclear localization. Successful knockdown of importin 1 was confirmed by western blot, and nuclear penetration by variant 13 was then compared in untransfected cells, cells transfected with control siRNA, and cells transfected with siRNA for importin β1. A significant reduction in variant 13 signal was evident in the importin 1 knockdown cells compared to cells with intact importin β1 (FIG. 2).

The finding that three different small molecule inhibitors of the importin pathway interfere with nuclear localization by variant 13, and that siRNA-mediated knockdown of importin 1 has a similar effect, strongly implicates the importin pathway is involved in the nucleocytoplasmic shuttling of variant 13.

### Example 7: Nuclear Penetration by ANAs in Human SLE Sera is Inhibited by Nuclear Import Inhibitors

Materials and Methods

SLE Patient Sera

The McGill University Health Centre SLE clinic maintains a registry of SLE patients who undergo annual assessment. The registry, including collection and analysis of the data and samples, has the ethical approval of the MUHC institutional review board. Twenty de-identified SLE serum samples meeting study criteria were randomly selected from the total available pool for further testing.

Study criteria were intended to maximize the homogeneity of the samples and the probability of identifying a sample containing nuclear-penetrating ANAs. Inclusion criteria specified that samples were from female patients with ANA positive disease and had high levels of anti-dsDNA antibodies. Samples from patients that were taking steroid or other immunosuppressive medications (other than hydroxychloroquine) at the time of collection were excluded due to concern that these medications could reduce the probability of detecting nuclear penetrating autoantibodies. Samples from patients with a concurrent diagnosis of cancer were also excluded due to concern that malignancy may perturb autoantibody profiles.

To test the serum samples for the presence of nuclear-penetrating ANAs, MCF7 cells were treated with each sample for one hour, followed by washing, fixation, and immunostaining for IgG. Serum samples were scored positive for nuclear-penetrating antibodies if they reproducibly yielded intranuclear staining in independent experiments. Of the twenty samples, samples SLE-4, -8, -9, and -19 tested positive and were used for experimental testing.

SLE Sera Nuclear Import

DLD1 cells were pre-treated with either control media, or media containing nuclear import inhibitors for four hours. Subsequent co-treatment with inhibitor and patient SLE serum (diluted 1:50) was performed for one hour at 37° C. Following treatment, cells were fixed, blocked and probed with an alkaline-phosphatase anti-human IgG primary antibody overnight (Fisher Scientific). Cell monolayers were washed, and color developed using NBT/BCIP reagent (Fisher Scientific) for the same duration across all experiments. Following chromogen color development, all monolayers were washed, and brightfield images acquired immediately on an Evos F microscope.

Results

Based on the apparent conservation of the bipartite NLS in nuclear-localizing anti-DNA autoantibodies in MRL/lpr mice, experiments were designed to determine if the nuclear-localizing autoantibodies in human SLE serum would use a similar method of nuclear penetration that is dependent on the importin pathway, and that ivermectin would therefore also inhibit their ability to penetrate nuclei. Twenty SLE serum samples, labeled SLE-1-20, were screened for the presence of nuclear penetrating ANAs (Alarcon-Segovia et al., Clin Exp Immunol 35, 364-375 (1979); Golan et al., J Invest Dermatol 100, 316-322 (1993)) by anti-IgG immunostaining of treated cells. Samples SLE-4, 8, 9, and 19 were selected for further use based on their reproducible yield of strong nuclear staining in both MCF7 and DLD1 cells, consistent with the presence of nuclear-penetrating ANAs.

The ability of ivermectin to inhibit penetration by the ANAs in SLE-4, 8, 9, and 19 into DLD1 cell nuclei was next tested. Pre- and co-treatment with ivermectin reduced the nuclear staining associated with each sample, consistent with inhibition of nuclear uptake of ANAs. In addition, mifepristone and importazole were similarly found to inhibit nuclear uptake of ANAs in SLE-19 in a dose-dependent manner.

These findings show that the nucleocytoplasmic shuttling of some SLE sera autoantibodies can be blocked by treatment with inhibitors of the importin pathway.

### Example 8: Nuclear Penetration by a Human Scleroderma Autoantibody is Inhibited by Ivermectin

In addition to SLE, cell-penetrating autoantibodies are detected in other autoimmune diseases such as scleroderma. A panel of human scleroderma autoantibodies was screened for nuclear penetration, and one nuclear-penetrating autoantibody, SSC, was selected for testing of the effect of ivermectin on its efficiency of nuclear uptake. SSC was expressed in and purified from HEK293T cells. Microscopic analysis revealed nuclear penetration of SSC into DLD1 cells and the effect of ivermectin. Ivermectin inhibited the nuclear uptake of SSC, confirming that ivermectin can modulate the nuclear-penetrating activity of autoantibodies from multiple autoimmune diseases.

Nuclear import of macromolecules occurs via an energy-dependent process through nuclear pore complexes (Fahrenkrog and Aebi, Nature Reviews Molecular Cell Biology 4, 757 (2003)), and an exposed NLS in macromolecules larger than 60 kDa facilitates interaction with nuclear import machinery (Freitas and Cunha, Current Genomics 10, 550-557 (2009)). Nuclear import via the importin pathway proceeds with the formation of an importin-cargo complex following recognition of a NLS motif by one of several importins. Subsequently, the cargo-importin complex is recruited to the nuclear pore complex, and gains entry into the nucleus. Interaction of RanGTP with the importin-cargo complex results in cargo dissociation from importin. Herein the role of the importin pathway in variant 13 nuclear penetration was explored through incubation with a panel of known nuclear import inhibitors, and also by siRNA-mediated importin β1 knockdown studies. Both approaches demonstrated significant blockade of variant 13 nuclear penetration, confirming the involvement of the nuclear import pathway in the nuclear uptake of variant 13.

NLS-like motifs within CDR regions of nuclear-localizing ANAs have previously been proposed to be involved in their mechanism of nuclear import (Im et al., Mol Immunol 67, 377-387 (2015); Im et al., Animal Cells and Systems 21, 382-387 (2017)). Herein a potential bipartite cNLS was identified in the 3E10 VL, and this sequence is conserved in several other nuclear-localizing anti-dsDNA autoantibodies.

The identification of a possible bipartite NLS in 3E10, potentially conserved in several other anti-dsDNA antibodies, including autoantibodies from lupus (SLE), combined with the finding that an inhibitors of the importin pathway (e.g., ivermectin) inhibit nuclear localization by variant 13 indicates that the importin pathway is involved in the mechanism of nuclear localization by 3E10, and perhaps the nuclear import of many ANAs, including pathogenic ANAs in human SLE serum. Consistent with this, ivermectin, a small molecule inhibitor of the importin α/β pathway, blocked the nuclear-penetrating activity of ANAs in each of the SLE serum samples tested in the disclosed studies, and also inhibited the uptake of a nuclear-penetrating scleroderma autoantibody.

It is known that ENT2 is involved in transport of 3E10 across the cell membrane, and above-discussed findings indicate that the bipartite NLS and importin pathway aid 3E10 translocation across the nuclear envelope. Because many proteins including DNA repair enzymes utilize the importin pathway for nuclear import, the findings raise the possibility that 3E10 may perturb the import of such proteins and this may further contribute to the effect of 3E10 on DNA repair and other intranuclear functions. Another proposed mechanism for 3E10 nuclear localization is exploitation of the nuclear import of these proteins during recruitment, through association with DNA damage repair proteins in the cytoplasm.

Ivermectin and mifepristone are FDA approved drugs with known safety records, and the discovery that these compounds inhibit nuclear penetration by variant 13 and by human SLE antibodies indicates that they and/or other inhibitors of nuclear import have potential to improve outcomes in patients with SLE or other autoimmune diseases in which nuclear-penetrating antibodies contribute to the disease process. In sum, the experiments identify the importin α/β pathway as a druggable gatekeeper for nuclear-localizing autoantibodies.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of treating an autoimmune disease comprising administering to a subject in need thereof an effective amount of an inhibitor of the importin pathway to reduce nuclear localization of nuclear penetrating antibodies in the subject.

2. The method of claim 1, wherein the autoimmune disease has one or more symptoms or pathology dependent on or otherwise caused by nuclear penetrating are antibodies.

3. The method of claim 1 or 2, wherein the nuclear penetrating antibodies autoantibodies.

4. The method of any one of claims 1 to 3, wherein the autoimmune disease is selected from the group consisting of systemic lupus erythematosus (lupus or SLE), systemic sclerosis (scleroderma), Graves' disease, myasthenia gravis, autoimmune hemolytic anemia, and pemphigus vulgaris, and additionally may contribute to the severity of disease in other autoimmune diseases such as rheumatoid arthritis, autoimmune thrombocytopenia, autoimmune atrophic gastritis of pernicious anemia, myasthenia gravis, Goodpasture's syndrome, diabetes mellitus, multiple sclerosis, Hashimoto's thyroiditis, Crohn's disease, and Sjögren's syndrome, primary biliary cirrhosis, primary sclerosing cholangitis, psoriasis, psoriatic arthritis, POEMS syndrome, dermatomyositis, inclusion body myositis, inflammatory myopathies, vasculitis syndromes including but not limited to Churg-Strauss Syndrome, Wegener granulomatosis, Behcet's disease, Buerger's disease, Kawasaki disease, Takayasu's arteritis, Henoch-Schonlein purpura, Giant cell arteritis, polyarteritis nodosa.

5. The method of any one of claims 1 to 4, wherein the autoimmune disease is a lupus.

6. The method of claim 5, wherein the lupus is selected from group consisting of systemic lupus erythematosus, discoid lupus erythematosus, subacute cutaneous lupus erythematosus, neonatal lupus, and drug-induced lupus.

7. The method of any one of claims 1 to 4, wherein the autoimmune disease is scleroderma.

8. A method of treating lupus comprising administering to a subject in need thereof effective amount of an inhibitor of the importin pathway to reduce one or more symptoms of the lupus.

9. The method of claim 8, wherein the lupus is selected from group consisting of systemic lupus erythematosus, discoid lupus erythematosus, subacute cutaneous lupus erythematosus, neonatal lupus, and drug-induced lupus.

10. A method of treating scleroderma comprising administering to a subject in need thereof effective amount of an inhibitor of the importin pathway to reduce one or more symptoms of the scleroderma.

11. A method of reducing penetration by an antibody into the nucleus of a cell comprising contacting the cell with an effective amount of an inhibitor of an importin pathway to reduce nuclear localization of the antibody.

12. The method of claim 11, wherein the antibodies comprise a nuclear localization signal.

13. The method of claim 11 or 12, wherein the antibody is an autoantibody.

14. The method of any one of claims 11-13, wherein the contacting occurs in vivo in subject in need thereof.

15. The method of claim 14, wherein the subject has an autoimmune disease.

16. The method of claim 14 or 15, wherein the subject has cancer.

17. The method of claim 16, wherein the subject is administered the antibody in an effective amount to inhibit DNA repair or directly damage DNA separately or together with the inhibitor of the importin pathway.

18. The method of claim 17, wherein the antibody is selected from the group consisting of 3E10, 5C6, fragments and fusions of 3E10 and 5C6, and variants and humanized forms of 3E10, 5C6, and fragments and fusions of 3E10 and 5C6.

19. A method of inhibiting nucleocytoplasmic shuttling of cellular proteins by contacting a cell with an antibody that penetrates nuclei via the importin pathway.

20. The method of claim 19, wherein an antibody that penetrates nuclei via the importin pathway inhibits nucleocytoplasmic shuttling of DNA repair factors including but not limited to RAD51.

21. A method of identifying the presence of importin-dependent nuclear-penetrating antibodies comprising contacting a test sample (for example serum, whole blood, CSF, pleural/pericardial fluid, or any other physiologic specimens) with an importin pathway protein, collecting the importin pathway protein, and detecting any antibodies bound thereto.

22. The method of any one of claims 1-18, wherein the inhibitor of the importin pathway inhibits an importin protein.

23. The method of any one of claims 1-18, wherein the inhibitor of the importin pathway disrupts a nucleoporin.

24. The method of any one of claims 1-18, wherein the inhibitor of the importin pathway inhibits a Ran protein.

25. The method of any one of claims 1-18, wherein the inhibitor of the importin pathway inhibits Ran GTPase activity.

26. The method of any one of claims 1-18, wherein the inhibitor of the importin pathway reduces cellular GTP content.

27. The method of any one of claims 1-18, wherein the inhibitor of the importin pathway inhibits a transportin protein.

28. The method of any one of claims 1-18, wherein the inhibitor of the importin pathway inhibits importin-α and/or importin-β.

29. The method of any one of claims 1-18, wherein the inhibitor of the importin pathway inhibits one or more of importin-4, importin-5, importin-7, importin-8, importin-9, importin-11, importin-13, importin-α1, importin-α2, importin-α3, importin-α4, importin-α5, importin-α6 and importin-β1, importin-β2, a nucleoporin, a Ran protein.

30. The method of any one of claims 1-18, wherein the inhibitor of the importin pathway inhibits expression of one or more of IPO4, IPO5, IPO7, IPO11, IPO13, KPNA1, KPNA2, KPNA3, KPNA4, KPNA5, KPNA6, KPNB1, TNPO1.

31. The method of any one of claims 1-18, wherein the inhibitor of the importin pathway is a molecule which is selected from the group consisting of a macrocyclic lactone, a diaminoquinazoline, a quinoxaline, a steroid, a peptide inhibitor, a binding protein, an antibody or fragment thereof, a peptidomimetic inhibitor, a retinoid or derivative thereof, and an oligonucleotide inhibitor.

32. The method of claim 31, wherein the inhibitor of the importin pathway is a macrocyclic lactone.

33. The method of claim 32, wherein the macrocyclic lactone is an avermectin or a milbemycin.

34. The method of claim 32 where in the macrocyclic lactone is selected from the group consisting of avermectin B₁ₐ/B_{1b} (abamectin), 22,23-dihydroavermectin B₁ₐ/B_{1b} (ivermectin), doramectin, moxidectin, dimadectin, emamectin, eprinomectin, latidectin, lepimectin, selamectin, milbemycin D, milbemectin, milbemycin B, milbemycin oxime, nemadectin, and combinations thereof.

35. The method of claim 31, wherein the inhibitor of the importin pathway is a diaminoquinazoline.

36. The method of claim 35, wherein the diaminoquinazoline is importazole.

37. The method of claim 31, wherein the inhibitor of the importin pathway is a steroid.

38. The method of claim 37, wherein the steroid is mifepristone.

39. The method of claim 31, wherein the inhibitor of the importin pathway is a retinoid.

40. The method of claim 31, wherein the inhibitor of the importin pathway is a peptide.

41. The method of claim 31, wherein the inhibitor of the importin pathway is a binding protein.

42. The method of claim 31, wherein the inhibitor of the importin pathway is an antibody or fragment thereof.

43. The method of claim 31, wherein the inhibitor of the importin pathway is an oligonucleotide inhibitor.

44. The method of claim 43, wherein the oligonucleotide inhibitor is an antisense RNA or DNA, siRNA or siDNA, miRNA, miRNA mimic, shRNA or DNA and Chimeric Antisense DNA or RNA.

45. The method claim 44, wherein the inhibitor of the importin pathway is an siRNA, shRNA, or miRNA.

46. The method of any one of claims 1-18 or 22-45 wherein the inhibitor is administered to a subject in need thereof by a parenteral, enteral, transdermal, or transmucosal route of administration.

47. A composition comprising an effective amount an inhibitor of any one of claims 22-44 to reduce nuclear localization of a nuclear penetrating antibody.

48. A composition comprising an effective amount an inhibitor of any one of claims 22-44 to reduce one or more symptoms of an autoimmune disease.

49. The composition of claim 47 or 48 wherein inhibitor is a macrocyclic lactone.

50. The composition of claim 49, wherein the macrocyclic lactone is selected from the group consisting of avermectin B₁ₐ/B_{1b} (abamectin), 22,23-dihydroavermectin B₁ₐ/B_{1b} (ivermectin), doramectin, moxidectin, dimadectin, emamectin, eprinomectin, latidectin, lepimectin, selamectin, milbemycin D, milbemectin, milbemycin B, milbemycin oxime, nemadectin, and combinations thereof.


SEQUENCE LISTING (69 sequences)

SEQ ID NO: 1 - protein, 111 aa, Mus musculus
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 2 - protein, 111 aa, Mus musculus
DIVLTQSPASLAVSLGQRATISCRASKSVSTSSYSYMHWYQQKPGQPPKLLIKYASYLESGVPARFSGSGSGTDFHLNIHPVEEEDAATYYCQHSREFPWTFGGGTKLELK

SEQ ID NO: 3 - protein, 15 aa, Artificial Sequence
  Synthetic Polypeptide
RASKSVSTSSYSYMH

SEQ ID NO: 4 - protein, 7 aa, Artificial Sequence
  Synthetic Polypeptide
YASYLES

SEQ ID NO: 5 - protein, 9 aa, Artificial Sequence
  Synthetic Polypeptide
QHSREFPWT

SEQ ID NO: 6 - protein, 116 aa, Mus musculus
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 7 - protein, 116 aa, Artificial Sequence
  Synthetic Polypeptide
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 8 - protein, 5 aa, Artificial Sequence
  Synthetic Polypeptide
DYGMH

SEQ ID NO: 9 - protein, 5 aa, Artificial Sequence
  Synthetic Polypeptide
NYGMH

SEQ ID NO: 10 - protein, 17 aa, Artificial Sequence
  Synthetic Polypeptide
YISSGSSTIYYADTVKG

SEQ ID NO: 11 - protein, 7 aa, Artificial Sequence
  Synthetic Polypeptide
RGLLLDY

SEQ ID NO: 12 - protein, 274 aa, Artificial Sequence
  Synthetic Polypeptide
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 13 - protein, 541 aa, Artificial Sequence
  Synthetic Polypeptide
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 14 - protein, 116 aa, Artificial Sequence
  Synthetic Polypeptide
EVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 15 - protein, 116 aa, Artificial Sequence
  Synthetic Polypeptide
EVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 16 - protein, 116 aa, Artificial Sequence
  Synthetic Polypeptide
EVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 17 - protein, 116 aa, Artificial Sequence
  Synthetic Polypeptide
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 18 - protein, 116 aa, Artificial Sequence
  Synthetic Polypeptide
EVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 19 - protein, 116 aa, Artificial Sequence
  Synthetic Polypeptide
EVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 20 - protein, 111 aa, Artificial Sequence
  Synthetic Polypeptide
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIK

SEQ ID NO: 21 - protein, 111 aa, Artificial Sequence
  Synthetic Polypeptide
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIK

SEQ ID NO: 22 - protein, 111 aa, Artificial Sequence
  Synthetic Polypeptide
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIK

SEQ ID NO: 23 - protein, 111 aa, Artificial Sequence
  Synthetic Polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 24 - protein, 111 aa, Artificial Sequence
  Synthetic Polypeptide
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIK

SEQ ID NO: 25 - protein, 111 aa, Artificial Sequence
  Synthetic Polypeptide
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIK

SEQ ID NO: 26 - protein, 515 aa, Artificial Sequence
  Synthetic Polypeptide
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 27 - protein, 515 aa, Artificial Sequence
  Synthetic Polypeptide
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 28 - protein, 515 aa, Artificial Sequence
  Synthetic Polypeptide
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 29 - protein, 515 aa, Artificial Sequence
  Synthetic Polypeptide
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 30 - protein, 515 aa, Artificial Sequence
  Synthetic Polypeptide
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 31 - protein, 515 aa, Artificial Sequence
  Synthetic Polypeptide
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 32 - protein, 515 aa, Artificial Sequence
  Synthetic Polypeptide
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 33 - protein, 515 aa, Artificial Sequence
  Synthetic Polypeptide
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 34 - protein, 515 aa, Artificial Sequence
  Synthetic Polypeptide
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 35 - protein, 515 aa, Artificial Sequence
  Synthetic Polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 36 - protein, 515 aa, Artificial Sequence
  Synthetic Polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 37 - protein, 515 aa, Artificial Sequence
  Synthetic Polypeptide
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 38 - protein, 515 aa, Artificial Sequence
  Synthetic Polypeptide
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 39 - protein, 515 aa, Artificial Sequence
  Synthetic Polypeptide
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 40 - protein, 515 aa, Artificial Sequence
  Synthetic Polypeptide
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 41 - protein, 515 aa, Artificial Sequence
  Synthetic Polypeptide
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 42 - protein, 112 aa, Mus musculus
DIVLTQSPASLAAVSLGERATISYRASKSVSTSGYSYMHWNQQKPGQAPRLLIYLVSNLESGVPARFSGSGSGTDFTLNIHPVEEEDAATYYCQHIRELDTFFGGGTKLEIK

SEQ ID NO: 43 - protein, 15 aa, Artificial Sequence
  Synthetic Polypeptide
RASKSVSTSGYSYMH

SEQ ID NO: 44 - protein, 7 aa, Artificial Sequence
  Synthetic Polypeptide
LVSNLES

SEQ ID NO: 45 - protein, 9 aa, Artificial Sequence
  Synthetic Polypeptide
QHIRELDTF

SEQ ID NO: 46 - protein, 120 aa, Mus musculus
QLKLVESGGGLVKPGGSLKLSCAASGFTFSSYTMSWVRQTPAKRLEWVATISSGGGSTYYPDSVKGRFTISRDNARNTLYLQMSSLRSEDTAMYYCARRAYSKRGAMDYWGQGTSVTVSS

SEQ ID NO: 47 - protein, 5 aa, Artificial Sequence
  Synthetic Polypeptide
SYTMS

SEQ ID NO: 48 - protein, 17 aa, Artificial Sequence
  Synthetic Polypeptide
TISSGGGSTYYPDSVKG

SEQ ID NO: 49 - protein, 11 aa, Artificial Sequence
  Synthetic Polypeptide
RAYSKRGAMDY

SEQ ID NO: 50 - protein, 15 aa, Artificial Sequence
  Synthetic Polypeptide
GGGGSGGGGSGGGGS

SEQ ID NO: 51 - protein, 7 aa, Artificial Sequence
  Synthetic Polypeptide
GQSSRSS

SEQ ID NO: 52 - protein, 18 aa, Artificial Sequence
  Synthetic Polypeptide
GQSSRSSSGGGSSGGGGS

SEQ ID NO: 53 - protein, 4 aa, Artificial Sequence
  Synthetic Polypeptide
GSGS

SEQ ID NO: 54 - protein, 4 aa, Artificial Sequence
  Synthetic Polypeptide
GGGS

SEQ ID NO: 55 - protein, 10 aa, Artificial Sequence
  Synthetic Polypeptide
GGGGSGGGGS

SEQ ID NO: 56 - protein, 20 aa, Artificial Sequence
  Synthetic Polypeptide
GGGGSGGGGSGGGGSGGGGS

SEQ ID NO: 57 - protein, 21 aa, Artificial Sequence
  Synthetic Polypeptide
RADAAPGGGGSGGGGSGGGGS

SEQ ID NO: 58 - protein, 19 aa, Artificial Sequence
  Synthetic Polypeptide
ASTKGPSVFPLAPLESSGS

SEQ ID NO: 59 - protein, 31 aa, Artificial Sequence
  Synthetic Polypeptide
RASKSVSTSSYSYMHWYQQKPGQPPKLLIKY

SEQ ID NO: 60 - protein, 31 aa, Artificial Sequence
  Synthetic Polypeptide
RASKTVSTSSYSYMHWYQQKPGQPPKLLIKY

SEQ ID NO: 61 - protein, 33 aa, Artificial Sequence
  Synthetic Polypeptide
RVTITCRASKSVSTSSYSYMHWYQQKPGKAPKL

SEQ ID NO: 62 - protein, 107 aa, Mus musculus
DIVLTQSPATLSVTPGDRVSLSCRASQSISNYLHWYQQKSHESPRLLIKYASQSISGIPSRFSGSGSGTDFTLSIISVETEDFGMYFCQQSNSWPLTFGAGTKLELK

SEQ ID NO: 63 - protein, 113 aa, Mus musculus
DVVMTQSPASLAVSLGQRATISCRASKSVSTSSYNYMHWHQQKPGQPPKLLIKYASYLESGVPARFSGSGSGTDFTLNIHPVEEEDAATYYCHHSREFPWTFGGGTKLEIKRA

SEQ ID NO: 64 - protein, 113 aa, Mus musculus
DLVMSQSPSSLAVSAGEKVTMSCKSSQSLFNSRTRKNYLAWYQQKPGQSPKLLIYWASTRESGVPDRFTGSGSGTDFTLTISSVQAEDLAVYYCKQSYYHMYTFGSGTKLEIK

SEQ ID NO: 65 - protein, 26 aa, Artificial Sequence
  Synthetic Polypeptide
RRRRPRKRPLEWDEDEEPPRKRKRLW

SEQ ID NO: 66 - protein, 26 aa, Artificial Sequence
  Synthetic Polypeptide
RRRRRRKRKREWDDDDDPPKKRRRLD

SEQ ID NO: 67 - protein, 38 aa, Artificial Sequence
  Synthetic Polypeptide
GGSYNDFGNYNNQSSNFGPMKGGNFGGRFEPYANPTKR

SEQ ID NO: 68 - protein, 31 aa, Artificial Sequence
  Synthetic Polypeptide
RASKSVSTSNYSYMYWYQQKPGQPPKLLIKY

SEQ ID NO: 69 - protein, 31 aa, Artificial Sequence
  Synthetic Polypeptide
RASKSVSTSSYNYIHWHQQKPGQPPKLLIKY